(19) United States
(12) Patent Application Publication
(10) Pub. No.: US 2008/0038730 A1
(43) Pub. Date: Feb. 14, 2008

(54) Diagnostic and Therapeutic Use of Kcnj6 for Alzheimer's Disease

(76) Inventors: Heinz Von der Kammer, Hamburg (DE); Johannes Pohlner, Hamburg (DE)

Correspondence Address: VENABLE LLP, P.O. BOX 34385, WASHINGTON, DC, 20043-9998, US

(21) Appl. No.: 11/596,128

(22) PCT Filed: May 9, 2005
(86) PCT No.: PCT/EP05/52084

§ 371 Date: Nov. 9, 2006

Related U.S. Application Data
(60) Provisional application No. 60/569,268, filed on May 10, 2004.

(51) Int. Cl. C12Q 1/68; A01K 67/00; G01N 33/00

(52) U.S. Cl. 435/6; 435/7.92; 800/12

(57) ABSTRACT

The present invention discloses a dysregulation of the KCNJ6 gene and the protein products thereof in Alzheimer's disease patients. Based on this finding, the invention provides a method for diagnosing and prognosticating Alzheimer's disease in a subject, and for determining whether a subject is at increased risk of developing Alzheimer's disease. Furthermore, this invention provides therapeutic and prophylactic methods for treating and preventing Alzheimer's disease and related neurodegenerative disorders using the KCNJ6 gene and its corresponding gene products. Screening methods for modulating agents of neurodegenerative diseases are also disclosed.

The present invention relates to methods of diagnosing, prognosticating and monitoring the progression of neurodegenerative diseases in a subject. Furthermore, methods of therapy control and screening for modulating agents of neurodegenerative diseases are provided. The invention also discloses pharmaceutical compositions, kits, and recombinant animal models.

Neurodegenerative diseases, in particular Alzheimer's disease (AD), have a strongly debilitating impact on a patient's life. Furthermore, these diseases constitute an enormous health, social, and economic burden. AD is the most common neurodegenerative disease, accounting for about 70% of all dementia cases, and it is probably the most devastating age-related neurodegenerative condition affecting about 10% of the population over 65 years of age and up to 45% over age 85 (for a recent review see Vickers et al., Progress in Neurobiology 2000, 60: 139-165). Presently, this amounts to an estimated 12 million cases in the US, Europe, and Japan. This situation will inevitably worsen with the demographic increase in the number of old people (“aging of the baby boomers”) in developed countries. The neuropathological hallmarks that occur in the brains of individuals with AD are senile plaques, composed of amyloid-β protein, and profound cytoskeletal changes coinciding with the appearance of abnormal filamentous structures and the formation of neurofibrillary tangles.

The amyloid-β protein evolves from the cleavage of the amyloid precursor protein (APP) by different kinds of proteases. The cleavage by the β/γ-secretase leads to the formation of Aβ peptides of different lengths, typically a short more soluble and slow aggregating peptide consisting of 40 amino acids and a longer 42 amino acid peptide, which rapidly aggregates outside the cells, forming the characteristic amyloid plaques (Selkoe, Physiological Rev 2001, 81: 741-66; Greenfield et al., Frontiers Bioscience 2000, 5: D72-83). Two types of plaques, diffuse plaques and neuritic plaques, can be detected in the brain of AD patients, the latter ones being the classical, most prevalent type. They are primarily found in the cerebral cortex and hippocampus. The neuritic plaques have a diameter of 50 μm to 200 μm and are composed of insoluble fibrillar amyloids, fragments of dead neurons, of microglia and astrocytes, and other components such as neurotransmitters, apolipoprotein E, glycosaminoglycans, α1-antichymotrypsin and others. The generation of toxic Aβ deposits in the brain starts very early in the course of AD, and it is discussed to be a key player for the subsequent destructive processes leading to AD pathology. The other pathological hallmarks of AD are neurofibrillary tangles (NFTs) and abnormal neurites, described as neuropil threads (Braak and Braak, Acta Neuropathol 1991, 82: 239-259). NFTs emerge inside neurons and consist of chemically altered tau, which forms paired helical filaments twisted around each other. Along the formation of NFTs, a loss of neurons can be observed (Johnson and Jenkins, J Alzheimers Dis 1996, 1: 38-58; Johnson and Hartigan, J Alzheimers Dis 1999, 1: 329-351). The appearance of neurofibrillary tangles and their increasing number correlates well with the clinical severity of AD (Schmitt et al., Neurology 2000, 55: 370-376).

AD is a progressive disease that is associated with early deficits in memory formation and ultimately leads to the complete erosion of higher cognitive function. The cognitive disturbances include among other things memory impairment, aphasia, agnosia and the loss of executive functioning. A characteristic feature of the pathogenesis of AD is the selective vulnerability of particular brain regions and subpopulations of nerve cells to the degenerative process. Specifically, the temporal lobe region and the hippocampus are affected early and more severely during the progression of the disease. On the other hand, neurons within the frontal cortex, occipital cortex, and the cerebellum remain largely intact and are protected from neurodegeneration (Terry et al., Annals of Neurology 1981, 10: 184-92).

The age of onset of AD may vary within a range of 50 years, with early-onset AD occurring in people younger than 65 years of age, and late-onset of AD occurring in those older than 65 years. About 10% of all AD cases suffer from early-onset AD, with only 1-2% being familial, inherited cases.

Currently, there is no cure for AD, nor is there an effective treatment to halt the progression of AD or even to diagnose AD ante-mortem with high probability. Several risk factors have been identified that predispose an individual to develop AD, among them most prominently the epsilon 4 allele of the three different existing alleles (epsilon 2, 3, and 4) of the apolipoprotein E gene (ApoE) (Strittmatter et al., Proc Natl Acad Sci USA 1993, 90: 1977-81; Roses, Ann NY Acad Sci 1998, 855: 738-43). Although there are rare examples of early-onset AD which have been attributed to genetic defects in the genes for amyloid precursor protein (APP) on chromosome 21, presenilin-1 on chromosome 14, and presenilin-2 on chromosome 1, the prevalent form of late-onset sporadic AD is of hitherto unknown etiologic origin. The late onset and complex pathogenesis of neurodegenerative disorders pose a formidable challenge to the development of therapeutic and diagnostic agents. It is crucial to expand the pool of potential drug targets and diagnostic markers. It is therefore an object of the present invention to provide insight into the pathogenesis of neurological diseases and to provide methods, materials, agents, compositions, and animal models which are suited inter alia for the diagnosis and development of a treatment of these diseases. This object has been solved by the features of the independent claims. The subclaims define preferred embodiments of the present invention.

The present invention is based on the detection and dysregulated, differential expression of a gene coding for a G-protein coupled inward rectifier potassium ion channel subfamily J member 6, KCNJ6, or ATP-sensitive potassium channel, also named K(ATP)2, or Kir3.2, BIR1 or GIRK2, and of the protein products of KCNJ6 in human Alzheimer's disease brain samples.

Functional potassium ion (K⁺) channels are transmembrane proteins which are composed of several subunits forming either homo- or heteromeric tetramers. Several structural classes of potassium channels are known to date. The ATP-sensitive potassium channels (K_{ATP}), the inward rectifier potassium channels (Kirs) and the G protein-coupled potassium channels belong to the two-transmembrane domain (2TM) containing proteins. Each subunit consists of two membrane-spanning helices (M1, M2) connected by a pore loop (P-loop) (Kubo et al., Nature 1993, 362: 127-133). The N-terminal and C-terminal ends are located cytoplasmically. The P-loop, which is responsible for potassium selectivity (known as P-region) is structurally highly conserved. A functional inward rectifier potassium channel is made up of four of the 2TM subunits. The channels are formed of either identical subunits (Kir1, Kir2 and Kir6 channels) or of similar subunits (Kir3 channels). The formation of heteromeric channels give rise to an enormous diversity and thus, to a broad spectrum of different channels with a wide range of physiological properties and unique capabilities (Minor, Current Opinion Structural Biology 2001, 11: 408-414; Miller, Genome Biology 2000, 1: 1-5).

One family of the potassium ion channels is composed of inwardly-rectifying potassium (Kir) channels which mediate flow of potassium ions across the cell membrane in action potential generation and stabilize the resting potential of the cell membrane, thereby regulating for instance neuronal excitability as well as the cardiac action potential. Potassium channels of the Kir family are capable of conducting more current when the potential is hyperpolarized than when it is depolarized. They conduct potassium currents more in the inward direction than outward. The potassium channels of the Kir3 family are G-protein activated, they are opened by binding of G-protein beta-gamma-subunits to their cytoplasmic domains (Kofuji et al., Proc. Natl. Acad. Science USA 1995, 95:6542-6546). In general, potassium channels are involved in a multitude of physiological functions and play a major role for the electrical activity in cardiac, neuronal and neurosecretory cells. Many neuronal disorders like familial neonatal convulsion, episodic ataxia, various forms of epilepsy are linked to mutations in potassium channels (Boland et al., Neuroscience 1999, 91: 1557-1564; Charlier et al., Nature Genetics 1998, 18: 53-55; Steinlein et al., Human Molecular Genetics 1997, 6: 943-947; Wallace et al., Nature Genetics 1998, 19: 366-370). The inwardly rectifying potassium channels Kir3 are expressed throughout the brain and in the heart. Their activity regulates neuronal excitability, synaptic transmission and modulate the heart rate. Thus, they play important and different roles in the nervous system, whereby neuronal function is affected by the ion channel properties itself, by their location and density in specific neuronal compartments, as well as by expression gradients across neuronal populations.

Liaou and coworkers reported about the coassembly of Kir3.2 and Kir3.1 forming a G-protein gated rectifier channel in neurons from the mouse (Liaou et al., J. Neuroscience 1996, 16: 7137-7150; Kofuji et al., Proc. Natl. Acad. Science USA 1995, 95:6542-6546) and further data indicate that both channels are expressed during neuronal differentiation in the cerebellum (Slesinger et al., Neuron 1996, 16: 321-331; Ferrer et al., J. Biology Chemistry 1995, 270: 26086-26091). A mutant Kir3.2 channel showed in mice a reduced sensitivity to muscarinic receptor activation (M2) and did not respond to G-protein alpha subunit. An increased sodium ion permeability together with a reduced sensitivity for potassium ions resulted in an altered membrane excitability (Navarro et al., Science 1996, 272: 1950-1953; Rossi et al., J. Neuroscience 1998, 18:3537-3547). A gain-of-function mutation in Kir3.2 leads to a transgenic mouse exhibiting a phenotype known as weaver phenotype. These mice suffering from ataxia, hyperactivity and tremor. A transgenic mouse lacking the Kir3.2 channel is morphologically indistinguishable from a wildtype mouse with a normal cerebellar development but with a susceptibility to develop seizures (Signorini et al., Proc. Natl. Acad. Science USA 1997, 94: 923-927). Because the transgenic weaver mice show cell loss in the substantia nigra and in the granule layer of the cerebellum (Patil et al., Nature Genetics 1995, 11: 126-129) which is similar to the nigral cell loss observed in patients with Parkinson's disease, a shared genetic defect in the weaver mouse and Parkinson's disease was discussed (Yamada et al., Brain Research 1990, 526: 303-307; Gaspar et al., Neuroscience 1994, 61: 293-305; Abraham et al., FASEB Journal 1999, 13: 1901-1907), but to date no mutation in the Kir3.2 gene from patients with Parkinson's disease could be detected (Bandmann et al., Neuroscience 1996, 72: 877-879). The mouse Kcnj6 (Genbank accession number NP_{—}034736.1) is highly homologous to the human sequence (Genbank accession number P48051) sharing more than 98% identity.

The Kir channels are grouped in a distinct family which consists of seven subfamilies. One subfamily the Kir3 family consists of the Kir3.1, Kir3.2, Kir3.3 and Kir3.4 subunits. The Kir3.2 (synonyms: BIR1, GIRK2, KATP2, hiGIRK2, KCNJ7, KCNJ6) subunit of 423 amino acids with a molecular weight of approximately 48.5 kDa, is encoded by the KCNJ6 gene, originally designated KCNJ7 by Tsaur et al. (Diabetes 1995, 44: 592-596), was cloned by Sakura and coworkers in 1995 (mRNA of 2598 nucleotides, Genbank accession number L78480; Protein ID: P48051; Database ID GDB: 1518525, U52153) (Sakura et al., FEBS Letter 1995, 367: 193-197). The KCNJ6 gene (Kir3.2) has been mapped to chromosome 21q22.13 (Ohira et al., Genome Research 1997, 7: 47-58). A relation of KCNJ6 (Kir3.2) with Alzheimer's disease to our today's knowledge has not been disclosed so far.

The singular forms “a”, “an”, and “the” as used herein and in the claims include plural reference unless the context dictates otherwise. For example, “a cell” means as well a plurality of cells, and so forth. The term “and/or” as used in the present specification and in the claims implies that the phrases before and after this term are to be considered either as alternatives or in combination. For instance, the wording “determination of a level and/or an activity” means that either only a level, or only an activity, or both a level and an activity are determined. The term “level” as used herein is meant to comprise a gage of, or a measure of the amount of, or a concentration of a transcription product, for instance an mRNA, or a translation product, for instance a protein or polypeptide. The term “activity” as used herein shall be understood as a measure for the ability of a transcription product or a translation product to produce a biological effect or a measure for a level of biologically active molecules. The term “activity” also refers to biological activity and/or pharmacological activity which refers to binding, antagonization, repression, blocking, neutralization or sequestration of a potassium channel or potassium channel subunit and which refers to activation, agonization, upregulation of a potassium channel or potassium channel subunit including but not limited to the novel potassium channel polypeptide of SEQ ID NO: 1. “Biological activity” includes but is not limited to the transmembrane transport of potassium ions and/or transmembrane potassium ion flow and/or the regulation thereof. “Pharmacological activity” includes but is not limited to the ability of a potassium channel or a potassium channel subunit to bind a ligand, a compound, an agent, a modulator and/or another potassium channel subunit. The terms “level” and/or “activity” as used herein further refer to gene expression levels or gene activity. Gene expression can be defined as the utilization of the information contained in a gene by transcription and translation leading to the production of a gene product. “Dysregulation” shall mean an upregulation or downregulation of gene expression. A gene product comprises either RNA or protein and is the result of expression of a gene. The amount of a gene product can be used to measure how active a gene is. The term “gene” as used in the present specification and in the claims comprises both coding regions (exons) as well as non-coding regions (e.g. non-coding regulatory elements such as promoters or enhancers, introns, leader and trailer sequences). The term “ORF” is an acronym for “open reading frame” and refers to a nucleic acid sequence that does not possess a stop codon in at least one reading frame and therefore can potentially be translated into a sequence of amino acids. “Regulatory elements” shall comprise inducible and non-inducible promoters, enhancers, operators, and other elements that drive and regulate gene expression. The term “fragment” as used herein is meant to comprise e.g. an alternatively spliced, or truncated, or otherwise cleaved transcription product or translation product. The term “derivative” as used herein refers to a mutant, or an RNA-edited, or a chemically modified, or otherwise altered transcription product, or to a mutant, or chemically modified, or otherwise altered translation product. For the purpose of clarity, a derivative transcript, for instance, refers to a transcript having alterations in the nucleic acid sequence such as single or multiple nucleotide deletions, insertions, or exchanges. A derivative translation product, for instance, may be generated by processes such as altered phosphorylation, or glycosylation, or acetylation, or lipidation, or by altered signal peptide cleavage or other types of maturation cleavage. These processes may occur post-translationally. The term “modulator” as used in the present invention and in the claims refers to a molecule capable of changing or altering the level and/or the activity of a gene, or a transcription product of a gene, or a translation product of a gene. A “modulator” refers to a molecule which has the capacity to either enhance or inhibit, thus to “modulate” a functional property of a potassium channel subunit or potassium channel, to “modulate” binding, antagonization, repression, blocking, neutralization or sequestration of a potassium channel or potassium channel subunit and to “modulate” activation, agonization and upregulation. “Modulation” will be also used to refer to the capacity to affect the biological activity of a cell. Preferably, a “modulator” is capable of changing or altering the biological activity of a transcription product or a translation product of a gene. Said modulation, for instance, may be an increase or a decrease in the biological activity and/or pharmacological activity, a change in binding characteristics, or any other change or alteration in the biological, functional, or immunological properties of said translation product of a gene. The terms “agent”, “reagent”, or “compound” refer to any substance, chemical, composition, or extract that have a positive or negative biological effect on a cell, tissue, body fluid, or within the context of any biological system, or any assay system examined. They can be agonists, antagonists, partial agonists or inverse agonists of a target. Such agents, reagents, or compounds may be nucleic acids, natural or synthetic peptides or protein complexes, or fusion proteins. They may also be antibodies, organic or anorganic molecules or compositions, small molecules, drugs and any combinations of any of said agents above. They may be used for testing, for diagnostic or for therapeutic purposes. The terms “oligonucleotide primer” or “primer” refer to short nucleic acid sequences which can anneal to a given target polynucleotide by hybridization of the complementary base pairs and can be extended by a polymerase. They may be chosen to be specific to a particular sequence or they may be randomly selected, e.g. they will prime all possible sequences in a mix. The length of primers used herein may vary from 10 nucleotides to 80 nucleotides. “Probes” are short nucleic acid sequences of the nucleic acid sequences described and disclosed herein or sequences complementary therewith. They may comprise full length sequences, or fragments, derivatives, isoforms, or variants of a given sequence. The identification of hybridization complexes between a “probe” and an assayed sample allows the detection of the presence of other similar sequences within that sample. As used herein, “homolog or homology” is a term used in the art to describe the relatedness of a nucleotide or peptide sequence to another nucleotide or peptide sequence, which is determined by the degree of identity and/or similarity between said sequences compared. In the art, the terms “identity” and “similarity” mean the degree of polypeptide or polynucleotide sequence relatedness which are determined by matching a query sequence and other sequences of preferably the same type (nucleic acid or protein sequence) with each other. Preferred computer program methods to calculate and determine “identity” and “similarity” include, but are not limited to GCG BLAST (Basic Local Alignment Search Tool) (Altschul et al., J. Mol. Biol. 1990, 215: 403-410; Altschul et al., Nucleic Acids Res. 1997, 25: 3389-3402; Devereux et al., Nucleic Acids Res. 1984, 12: 387), BLASTN 2.0 (Gish W., http://blast.wustl.edu, 1996-2002), FASTA (Pearson and Lipman, Proc. Natl. Acad. Sci. USA 1988, 85: 2444-2448), and GCG GelMerge which determines and aligns a pair of contigs with the longest overlap (Wilbur and Lipman, SIAM J. Appl. Math. 1984, 44: 557-567; Needleman and Wunsch, J. Mol. Biol. 1970, 48: 443-453). The term “variant” as used herein refers to any polypeptide or protein, in reference to polypeptides and proteins disclosed in the present invention, in which one or more amino acids are added and/or substituted and/or deleted and/or inserted at the N-terminus, and/or the C-terminus, and/or within the native amino acid sequences of the native polypeptides or proteins of the present invention, but retains its essential properties. Furthermore, the term “variant” shall include any shorter or longer version of a polypeptide or protein. “Variants” shall also comprise a sequence that has at least about 80% sequence identity, more preferably at least about 90% sequence identity, and most preferably at least about 95% sequence identity with the amino acid sequences of KCNJ6 protein, SEQ ID NO: 1 (Kir3.2). “Variants” include, for example, proteins with conservative amino acid substitutions in highly conservative regions. “Proteins and polypeptides” of the present invention include variants, fragments and chemical derivatives of the protein comprising the amino acid sequences of KCNJ6 protein, SEQ ID NO: 1 (Kir3.2). Sequence variations shall be included wherein a codon are replaced with another codon due to alternative base sequences, but the amino acid sequence translated by the DNA sequence remains unchanged. This known in the art phenomenon is called redundancy of the set of codons which translate specific amino acids. Included shall be such exchange of amino acids which would have no effect on functionality, such as arginine for lysine, valine for leucine, asparagine for glutamine. Proteins and polypeptides can be included which can be isolated from nature or be produced by recombinant and/or synthetic means. Native proteins or polypeptides refer to naturally-occurring truncated or secreted forms, naturally occurring variant forms (e.g. splice-variants) and naturally occurring allelic variants. The term “isolated” as used herein is considered to refer to molecules or substances which have been changed and/or that are removed from their natural environment, i.e. isolated from a cell or from a living organism in which they normally occur, and that are separated or essentially purified from the coexisting components with which they are found to be associated in nature. This notion further means that the sequences encoding such molecules can be linked by the hand of man to polynucleotides, to which they are not linked in their natural state and such molecules can be produced by recombinant and/or synthetic means, it is also said that they are “non-native”. Even if for said purposes those sequences may be introduced into living or non-living organisms by methods known to those skilled in the art, and even if those sequences are still present in said organisms, they are still considered to be isolated. In the present invention, the terms “risk”, “susceptibility”, and “predisposition” are tantamount and are used with respect to the probability of developing a neurodegenerative disease, preferably Alzheimer's disease. The term “AD” shall mean Alzheimer's disease. “AD-type neuropathology” as used herein refers to neuropathological, neurophysiological, histopathological and clinical hallmarks as described in the instant invention and as commonly known from state-of-the-art literature (see: Iqbal, Swaab, Winblad and Wisniewski, Alzheimer's Disease and Related Disorders (Etiology, Pathogenesis and Therapeutics), Wiley & Sons, New York, Weinheim, Toronto, 1999; Scinto and Daffner, Early Diagnosis of Alzheimer's Disease, Humana Press, Totowa, N.J., 2000; Mayeux and Christen, Epidemiology of Alzheimer's Disease: From Gene to Prevention, Springer Press, Berlin, Heidelberg, New York, 1999; Younkin, Tanzi and Christen, Presenilins and Alzheimer's Disease, Springer Press, Berlin, Heidelberg, New York, 1998). The term “Braak stage” or “Braak staging” refers to the classification of brains according to the criteria proposed by Braak and Braak (Braak and Braak, Acta Neuropathology 1991, 82: 239-259). On the basis of the distribution of neurofibrillary tangles and neuropil threads, the neuropathologic progression of AD is divided into six stages (stage 0 to 6). In the instant invention Braak stages 0 to 2 represent healthy control persons (“controls”), and Braak stages 4 to 6 represent persons suffering from Alzheimer's disease (“AD patients”). The values obtained from said “controls” are the “reference values” representing a “known health status” and the values obtained from said “AD patients” are the “reference values” representing a “known disease status”. Braak stage 3 (middle Braak stage) may represent either a healthy control persons or an AD patient. The higher the Braak stage the more likely is the possibility to display the symptoms of AD. For a neuropathological assessment, i.e. an estimation of the probability that pathological changes of AD are the underlying cause of dementia, a recommendation is given by Braak H. (www. alzforum.org).

Neurodegenerative diseases or disorders according to the present invention comprise Alzheimer's disease, Parkinson's disease, Huntington's disease, amyotrophic lateral sclerosis, Pick's disease, fronto-temporal dementia, progressive nuclear palsy, corticobasal degeneration, cerebro-vascular dementia, multiple system atrophy, argyrophilic grain dementia and other tauopathies, and mild-cognitive impairment. Further conditions involving neurodegenerative processes are, for instance, ischemic stroke, age-related macular degeneration, narcolepsy, motor neuron diseases, prion diseases, traumatic nerve injury and repair, and multiple sclerosis.

The present invention discloses the identification, the differential expression, the differential regulation, a dysregulation of a gene coding for an ATP-sensitive potassium channel, a G-protein coupled inward rectifier potassium ion channel of the subfamily J, KCNJ6 (alias Kir3.2 or GIRK2), and of the protein products of said gene KCNJ6 (alias Kir3.2, GIRK2), in specific samples, in specific brain regions of AD patients in comparison with each other and/or in comparison to healthy age-matched control persons. The present invention discloses that the gene expression for KCNJ6 (Kir3.2) is varied, is dysregulated in AD-affected brains, in that KCNJ6 (Kir3.2) mRNA levels are decreased, are down-regulated in the temporal cortex as compared to the frontal cortex, or are elevated, are up-regulated in the frontal cortex as compared to the temporal cortex. Further, the present invention discloses that the KCNJ6 (Kir3.2) expression differs between the frontal cortex and the temporal cortex of healthy age-matched control subjects compared to the frontal cortex and the temporal cortex of AD patients. No such dysregulation is observed comparing samples with each other obtained from age-matched, healthy controls. This dysregulation presumably relates to a pathologic alteration of KCNJ6 (Kir3.2) in AD-affected brains. To date, no experiments have been described that demonstrate a relationship between the dysregulation of KCNJ6 (Kir3.2) gene expression and the pathology of neurodegenerative diseases, in particular AD. Likewise, no mutations in the KCNJ6 (Kir3.2) gene have been described to be associated with said diseases. Linking the KCNJ6 (Kir3.2) gene to such diseases offers new ways, inter alia, for the diagnosis and treatment of said diseases.

The present invention discloses a dysregulation of a gene coding for KCNJ6 (Kir3.2) in specific brain regions of AD patients. Neurons within the inferior temporal lobe, the entorhinal cortex, the hippocampus, and the amygdala are subject to degenerative processes in AD (Terry et al., Annals of Neurology 1981, 10:184-192). These brain regions are mostly involved in the processing of learning and memory functions and display a selective vulnerability to neuronal loss and degeneration in AD. In contrast, neurons within the frontal cortex, the occipital cortex, and the cerebellum remain largely intact and preserved from neurodegenerative processes. Brain tissues from the frontal cortex (F), the temporal cortex (T) of AD patients and healthy, age-matched control individuals were used for the herein disclosed examples. Consequently, the KCNJ6 (Kir3.2) gene and its corresponding transcription and/or translation products have a causative role in the regional selective neuronal degeneration typically observed in AD. Alternatively, KCNJ6 (Kir3.2) may confer a neuroprotective function to the remaining surviving nerve cells. Based on these disclosures, the present invention has utility for the diagnostic evaluation and prognosis as well as for the identification of a predisposition to a neurodegenerative disease, in particular AD. Furthermore, the present invention provides methods for the diagnostic monitoring of patients undergoing treatment for such a disease.

In one aspect, the invention features a method of diagnosing or prognosticating a neurodegenerative disease in a subject, or determining whether a subject is at increased risk of developing said disease. The method comprises: determining a level, or an activity, or both said level and said activity of (i) a transcription product of the gene coding for KCNJ6 protein, and/or of (ii) a translation product of the gene coding for KCNJ6 protein, and/or of (iii) a fragment, or derivative, or variant of said transcription or translation product in a sample obtained from said subject and comparing said level, and/or said activity of said transcription product and/or said translation product to a reference value representing a known disease status and/or to a reference value representing a known health status (healthy control), and said level and/or said activity is varied, is altered compared to a reference value representing a known health status, and/or is similar or equal to a reference value representing a known disease status, thereby diagnosing or prognosticating said neurodegenerative disease in said subject, or determining whether said subject is at increased risk of developing said neurodegenerative disease. The wording “in a subject” refers to results of the methods disclosed as far as they relate to a disease afflicting a subject, that is to say, said disease being “in” a subject.

The invention also relates to the construction and the use of primers and probes which are unique to the nucleic acid sequences, or fragments, or variants thereof, as disclosed in the present invention. The oligonucleotide primers and/or probes can be labeled specifically with fluorescent, bioluminescent, magnetic, or radioactive substances. The invention further relates to the detection and the production of said nucleic acid sequences, or fragments and variants thereof, using said specific oligonucleotide primers in appropriate combinations. PCR-analysis, a method well known to those skilled in the art, can be performed with said primer combinations to amplify said gene specific nucleic acid sequences from a sample containing nucleic acids. Such sample may be derived either from healthy or diseased subjects. Whether an amplification results in a specific nucleic acid product or not, and whether a fragment of different length can be obtained or not, may be indicative for a neurodegenerative disease, in particular Alzheimer's disease. Thus, the invention provides nucleic acid sequences, oligonucleotide primers, and probes of at least 10 bases in length up to the entire coding and gene sequences, useful for the detection of gene mutations and single nucleotide polymorphisms in a given sample comprising nucleic acid sequences to be examined, which may be associated with neurodegenerative diseases, in particular Alzheimer's disease. This feature has utility for developing rapid DNA-based diagnostic tests, preferably also in the format of a kit. Primers for KCNJ6 are exemplarily described in Example (iv).

In a further aspect, the invention features a method of monitoring the progression of a neurodegenerative disease in a subject. A level, or an activity, or both said level and said activity, of (i) a transcription product of the gene coding for KCNJ6 protein, and/or of (ii) a translation product of the gene coding for KCNJ6 protein, and/or of (iii) a fragment, or derivative, or variant of said transcription or translation product in a sample obtained from said subject is determined. Said level and/or said activity is compared to a reference value representing a known disease or health status. Thereby, the progression of said neurodegenerative disease in said subject is monitored.

In still a further aspect, the invention features a method of evaluating a treatment for a neurodegenerative disease, comprising determining a level, or an activity, or both said level and said activity of (i) a transcription product of the gene coding for KCNJ6 protein, and/or of (ii) a translation product of the gene coding for KCNJ6 protein, and/or of (iii) a fragment, or derivative, or variant of said transcription or translation product in a sample obtained from a subject being treated for said disease. Said level, or said activity, or both said level and said activity are compared to a reference value representing a known disease or health status, thereby evaluating the treatment for said neurodegenerative disease.

In a preferred embodiment of the herein claimed methods, kits, recombinant animals, molecules, assays, and uses of the instant invention, said KCNJ6 (Kir3.2) gene and proteins, also referred to as G-protein coupled inward rectifier potassium ion channel subfamily J member 6, formerly KCNJ7, or ATP-sensitive potassium channel, also named K(ATP)2, or Kir3.2, BIR1 or GIRK2, is represented by the KCNJ6 gene coding for the protein of SEQ ID NO: 1 (Kir3.2) (Genbank accession number P48051). The amino acid sequence of said protein is deduced from the mRNA sequence corresponding to SEQ ID NO: 2 (Kir3.2 cDNA) which corresponds to the cDNA sequence of Genbank accession number U52153 (Kir3.2). In the instant invention KCNJ6 also refers to the nucleic acid sequence of SEQ ID NO: 2, coding for the protein of SEQ ID NO: 1 (Genbank accession number P48051) and to the nucleic acid sequence SEQ ID NO: 4 representing the coding sequence (cds) of human KCNJ6. In the instant invention said sequences are “isolated” as the term is employed herein. Further, in the instant invention, the gene coding for said KCNJ6 protein is also generally referred to as the KCNJ6 gene or the Kir3.2 gene, or simply KCNJ6 or Kir3.2. The protein of KCNJ6 or Kir3.2 are also generally referred to as the KCNJ6 protein or Kir3.2 protein.

In a further preferred embodiment of the herein claimed methods, kits, recombinant animals, molecules, assays, and uses of the instant invention, said neurodegenerative disease or disorder is Alzheimer's disease, and said subjects suffer from Alzheimer's disease.

It is preferred that the sample to be analyzed and determined is selected from the group comprising brain tissue or other tissues, or body cells. The sample can also comprise cerebrospinal fluid or other body fluids including saliva, urine, blood, serum plasma, or mucus. Preferably, the methods of diagnosis, prognosis, monitoring the progression or evaluating a treatment for a neurodegenerative disease, according to the instant invention, can be practiced ex corpore, and such methods preferably relate to samples, for instance, body fluids or cells, removed, collected, or isolated from a subject or patient or healthy control person.

In further preferred embodiments, said reference value is that of a level, or an activity, or both said level and said activity of (i) a transcription product of the gene coding for KCNJ6 protein, and/or of (ii) a translation product of the gene coding for KCNJ6 protein, and/or of (iii) a fragment, or derivative, or variant of said transcription or translation product in a sample obtained from a subject not suffering from said neurodegenerative disease (healthy control person, control sample, control) or in a sample obtained from a subject suffering from a neurodegenerative disease, in particular Alzheimer's disease (patient sample, patient).

In preferred embodiments, an alteration in the level and/or activity of a transcription product of the gene coding for KCNJ6 protein and/or of a translation product of the gene coding for KCNJ6 protein and/or of a fragment, or derivative, or variant thereof in a sample cell, or tissue, or body fluid from said subject relative to a reference value representing a known health status (control sample) indicates a diagnosis, or prognosis, or increased risk of becoming diseased with a neurodegenerative disease, particularly AD.

In further preferred embodiments, an equal or similar level and/or activity of a transcription product of the gene coding for a KCNJ6 protein and/or of a translation product of the gene coding for a KCNJ6 protein and/or of a fragment, or derivative, or variant thereof in a sample cell, or tissue, or body fluid obtained from a subject relative to a reference value representing a known disease status of a neurodegenerative disease, in particular Alzheimer's disease (AD patient sample), indicates a diagnosis, or prognosis, or increased risk of becoming diseased with said neurodegenerative disease.

In preferred embodiments, measurement of the level of transcription products of the gene coding for KCNJ6 protein is performed in a sample obtained from a subject using a quantitative PCR-analysis with primer combinations to amplify said gene specific sequences from cDNA obtained by reverse transcription of RNA extracted from a sample of a subject. Primer combinations (SEQ ID NO: 5, SEQ ID NO: 6) are given in Example (iv) of the instant invention, but also other primers generated from the sequences as disclosed in the instant invention can be used. A Northern blot with probes specific for said gene can also be applied. It might further be preferred to measure transcription products by means of chip-based microarray technologies. These techniques are known to those of ordinary skill in the art (see Sambrook and Russell, Molecular Cloning: A Laboratory Manual, Cold Spring Harbor Laboratory Press, Cold Spring Harbor, N.Y., 2001; Schena M., Microarray Biochip Technology, Eaton Publishing, Natick, Mass., 2000). An example of an immunoassay is the detection and measurement of enzyme activity as disclosed and described in the patent application WO 02/14543.

Furthermore, a level and/or an activity of a translation product of the gene coding for KCNJ6 protein and/or of a fragment, or derivative, or variant of said translation product, and/or the level of activity of said translation product, and/or of a fragment, or derivative, or variant thereof, can be detected using an immunoassay, an activity assay, and/or a binding assay. These assays can measure the amount of binding between said protein molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g. ELISAs, Western blots and other techniques known to those of ordinary skill in the art (see Harlow and Lane, Antibodies: A Laboratory Manual, Cold Spring Harbor Laboratory Press, Cold Spring Harbor, N.Y., 1999 and Edwards R, Immunodiagnostics: A Practical Approach, Oxford University Press, Oxford; England, 1999). All these detection techniques may also be employed in the format of microarrays, protein-arrays, antibody microarrays, tissue microarrays, electronic biochip or protein-chip based technologies (see Schena M., Microarray Biochip Technology, Eaton Publishing, Natick, Mass., 2000).

In a preferred embodiment, the level, or the activity, or both said level and said activity of (i) a transcription product of the gene coding for KCNJ6 protein, and/or of (ii) a translation product of the gene coding KCNJ6 protein, and/or of (iii) a fragment, or derivative, or variant of said transcription or translation product in a series of samples taken from said subject over a period of time is compared, in order to monitor the progression of said disease. In further preferred embodiments, said subject receives a treatment prior to one or more of said sample gatherings. In yet another preferred embodiment, said level and/or activity is determined before and after said treatment of said subject.

In another aspect, the invention features a kit for diagnosing or prognosticating neurodegenerative diseases, in particular AD, in a subject, or determining the propensity or predisposition of a subject to develop a neurodegenerative disease, in particular AD, said kit comprising:

(a) at least one reagent which is selected from the group consisting of (i) reagents that selectively detect a transcription product of the gene coding for KCNJ6 protein (ii) reagents that selectively detect a translation product of the gene coding for KCNJ6 protein; and

(b) instructions for diagnosing, or prognosticating a neurodegenerative disease, in particular AD, or determining the propensity or predisposition of a subject to develop such a disease by

detecting a level, or an activity, or both said level and said activity, of said transcription product and/or said translation product of the gene coding for KCNJ6 protein, in a sample obtained from said subject; and
diagnosing or prognosticating a neurodegenerative disease, in particular AD, or determining the propensity or predisposition of said subject to develop such a disease, wherein a varied or altered level, or activity, or both said level and said activity, of said transcription product and/or said translation product compared to a reference value representing a known health status (control) and/or wherein a level, or activity, or both said level and said activity, of said transcription product and/or said translation product is similar or equal to a reference value representing a known disease status, preferably a disease status of AD, indicates a diagnosis or prognosis of a neurodegenerative disease, in particular AD, or an increased propensity or predisposition of developing such a disease. The kit, according to the present invention, may be particularly useful for the identification of individuals that are at risk of developing a neurodegenerative disease, in particular AD.

In a further aspect the invention features the use of a kit in a method of diagnosing or prognosticating a neurodegenerative disease, in particular Alzheimer's disease, in a subject, and in a method of determining the propensity or predisposition of a subject to develop such a disease by the steps of: (i) detecting in a sample obtained from said subject a level, or an activity, or both said level and said activity of a transcription product and/or of a translation product of a gene coding for KCNJ6, and (ii) comparing said level or activity, or both said level and said activity of a transcription product and/or of a translation product of a gene coding for KCNJ6 to a reference value representing a known health status and/or to a reference value representing a known disease status, and said level, or activity, or both said level and said activity, of said transcription product and/or said translation product is varied compared to a reference value representing a known health status, and/or is similar or equal to a reference value representing a known disease status.

Consequently, the kit, according to the present invention, may serve as a means for targeting identified individuals for early preventive measures or therapeutic intervention prior to disease onset, before irreversible damage in the course of the disease has been inflicted. Furthermore, in preferred embodiments, the kit featured in the invention is useful for monitoring a progression of a neurodegenerative disease, in particular AD in a subject, as well as monitoring success or failure of therapeutic treatment for such a disease of said subject.

In another aspect, the invention features a method of treating or preventing a neurodegenerative disease, in particular AD, in a subject comprising the administration to said subject in a therapeutically or prophylactically effective amount of an agent or agents which directly or indirectly affect a level, or an activity, or both said level and said activity, of (i) the gene coding for KCNJ6 protein, and/or (ii) a transcription product of the gene coding for KCNJ6 protein, and/or (iii) a translation product of the gene coding for KCNJ6 protein, and/or (iv) a fragment, or derivative, or variant of (i) to (iii). Said agent may comprise a small molecule, or it may also comprise a peptide, an oligopeptide, or a polypeptide. Said peptide, oligopeptide, or polypeptide may comprise an amino acid sequence of a translation product of the gene coding for KCNJ6 protein, or a fragment, or derivative, or a variant thereof. An agent for treating or preventing a neurodegenerative disease, in particular AD, according to the instant invention, may also consist of a nucleotide, an oligonucleotide, or a polynucleotide. Said oligonucleotide or polynucleotide may comprise a nucleotide sequence of the gene coding for KCNJ6 protein, either in sense orientation or in antisense orientation.

In preferred embodiments, the method comprises the application of per se known methods of gene therapy and/or antisense nucleic acid technology to administer said agent or agents. In general, gene therapy includes several approaches: molecular replacement of a mutated gene, addition of a new gene resulting in the synthesis of a therapeutic protein, and modulation of endogenous cellular gene expression by recombinant expression methods or by drugs. Gene-transfer techniques are described in detail (see e.g. Behr, Acc Chem Res 1993, 26: 274-278 and Mulligan, Science 1993, 260: 926-931) and include direct gene-transfer techniques such as mechanical microinjection of DNA into a cell as well as indirect techniques employing biological vectors (like recombinant viruses, especially retroviruses) or model liposomes, or techniques based on transfection with DNA coprecipitation with polycations, cell membrane pertubation by chemical (solvents, detergents, polymers, enzymes) or physical means (mechanic, osmotic, thermic, electric shocks). The postnatal gene transfer into the central nervous system has been described in detail (see e.g. Wolff, Curr Opin Neurobiol 1993, 3: 743-748).

In particular, the invention features a method of treating or preventing a neurodegenerative disease by means of antisense nucleic acid therapy, i.e. the down-regulation of an inappropriately expressed or defective gene by the introduction of antisense nucleic acids or derivatives thereof into certain critical cells (see e.g. Gillespie, DN&P 1992, 5: 389-395; Agrawal and Akhtar, Trends Biotechnol 1995, 13: 197-199; Crooke, Biotechnology 1992, 10: 882-6). Apart from hybridization strategies, the application of ribozymes, i.e. RNA molecules that act as enzymes, destroying RNA that carries the message of disease has also been described (see e.g. Barinaga, Science 1993, 262: 1512-1514). In preferred embodiments, the subject to be treated is a human, and therapeutic antisense nucleic acids or derivatives thereof are directed against transcription products of the gene coding for KCNJ6 protein. It is preferred that cells of the central nervous system, preferably the brain, of a subject are treated in such a way. Cell penetration can be performed by known strategies such as coupling of antisense nucleic acids and derivatives thereof to carrier particles, or the above described techniques. Strategies for administering targeted therapeutic oligo-deoxynucleotides are known to those of skill in the art (see e.g. Wickstrom, Trends Biotechnol 1992, 10: 281-287). In some cases, delivery can be performed by mere topical application. Further approaches are directed to intracellular expression of antisense RNA. In this strategy, cells are transformed ex vivo with a recombinant gene that directs the synthesis of an RNA that is complementary to a region of target nucleic acid. Therapeutical use of intracellularly expressed antisense RNA is procedurally similar to gene therapy. A recently developed method of regulating the intracellular expression of genes by the use of double-stranded RNA, known variously as RNA interference (RNAi), can be another effective approach for nucleic acid therapy (Hannon, Nature 2002, 418: 244-251).

In a further preferred embodiment, a method to investigate the effects of compounds and/or agents on a potassium channel formed by Kir3.2 subunits or on a heteromeric potassium channel formed by Kir3.2 subunits and Kir3.1 and/or Kir3.3 subunits and/or other potassium channel subunits, is provided. Thereby, the electrophysiological effect of compounds and/or agents on the potassium current mediated by Kir3.2 expressed alone or coexpressed with Kir3.1 and/or Kir3.3 and/or another potassium channel in appropriate cells, for example CHO-K1 cells, COS-7 cells or HEK293 cells, or in other neuronal cell lines, is examined. To conduct said examination the cDNA coding for human gene product Kir3.2 is cloned into an appropriate expression-vector. The cDNA coding for Kir3.1 and/or Kir3.3 and/or for another potassium channel, is cloned into another appropriate expression-vector. Appropriate cell lines, as mentioned above, are transfected with said plasmids, preferably using a reagent like DMRIE-C (liposome formulation of the cationic lipid 1,2-dimyristyloxypropyl-3-dimethyl-hydroxy ethyl ammonium bromide-chloesterol). Patch-clamp experiments can be performed in the voltage-clamp mode (Hamill et al., Pflügers Arch. 1981, 391: 85-100), and whole-cell currents will be recorded, and the obtained signals will be amplified, digitized, stored and analyzed using an appropriate software, for example Pulse/Pulsefit (HEKA, Lambrecht, Germany). If current “run-down” or “run-up” (Varnum et al., Pro. Natl. Acad. Sci. USA 1993, 90: 11528-11532) remains to be too strong for compound and/or agent effect evaluation, investigations on the mediated currents of said potassium channels can be performed with the perforated patch-clamp method to prevent unspecific current amplitude changes (Dart et al., J. Physiol. 1995, 483: 29-39; Dinesh & Hablitz, Brain Res. 1990, 535: 318-322). An example of a stimulation protocol for the investigation of the effects and reversibility of test compounds on Kir3.2 alone or coexpressed with Kir 3.1 and/or Kir3.3 and/or another potassium channel, is given below. Cells will be clamped at a holding potential of e.g. −60 mV. The pulse cycling rate may be 15 sec. For the compound and/or agent testing, stably transfected cells can be hyperpolarized from a holding potential of e.g. −50 mV for e.g. 100 msec or 250 msec or 500 msec to e.g. −160 mV in −10 or −20 mV increments, followed by, for instance, a 1 s depolarization to +90 mV. The current amplitude at the end of the test pulse to +90 mV will be used for the analysis. The method is also suitable to identify and test compounds and/or agents which are capable for opening, closing, activating, inactivating, or modifying the biophysical properties of Kir3.2 alone or coexpressed with Kir3.1 and/or Kir3.3 and/or another potassium channel. Modulators of potassium channels, in particular of inwardly rectifying potassium channels, thus identified and tested, can potentially influence learning and memory functions and can be used for therapeutic approaches, for example for neurodegenerative diseases, in particular for Alzheimer's disease.

In further preferred embodiments, the method comprises grafting donor cells into the central nervous system, preferably the brain, of said subject, or donor cells preferably treated so as to minimize or reduce graft rejection, wherein said donor cells are genetically modified by insertion of at least one transgene encoding said agent or agents. Said transgene might be carried by a viral vector, in particular a retroviral vector. The transgene can be inserted into the donor cells by a nonviral physical transfection of DNA encoding a transgene, in particular by microinjection. Insertion of the transgene can also be performed by electroporation, chemically mediated transfection, in particular calcium phosphate transfection or liposomal mediated transfection (see Mc Celland and Pardee, Expression Genetics: Accelerated and High-Throughput Methods, Eaton Publishing, Natick, Mass., 1999).

In preferred embodiments, said agent for treating and preventing a neurodegenerative disease, in particular AD, is a therapeutic protein which can be administered to said subject, preferably a human, by a process comprising introducing subject cells into said subject, said subject cells having been treated in vitro to insert a DNA segment encoding said therapeutic protein, said subject cells expressing in vivo in said subject a therapeutically effective amount of said therapeutic protein. Said DNA segment can be inserted into said cells in vitro by a viral vector, in particular a retroviral vector.

Methods of treatment, according to the present invention, comprise the application of therapeutic cloning, transplantation, and stem cell therapy using embryonic stem cells or embryonic germ cells and neuronal adult stem cells, combined with any of the previously described cell- and gene therapeutic methods. Stem cells may be totipotent or pluripotent. They may also be organ-specific. Strategies for repairing diseased and/or damaged brain cells or tissue comprise (i) taking donor cells from an adult tissue. Nuclei of those cells are transplanted into unfertilized egg cells from which the genetic material has been removed. Embryonic stem cells are isolated from the blastocyst stage of the cells which underwent somatic cell nuclear transfer. Use of differentiation factors then leads to a directed development of the stem cells to specialized cell types, preferably neuronal cells (Lanza et al., Nature Medicine 1999, 9: 975-977), or (ii) purifying adult stem cells, isolated from the central nervous system, or from bone marrow (mesenchymal stem cells), for in vitro expansion and subsequent grafting and transplantation, or (iii) directly inducing endogenous neural stem cells to proliferate, migrate, and differentiate into functional neurons (Peterson DA, Curr. Opin. Pharmacol. 2002, 2: 34-42) Adult neural stem cells are of great potential for repairing damaged or diseased brain tissues, as the germinal centers of the adult brain are free of neuronal damage or dysfunction (Colman A, Drug Discovery World 2001, 7: 66-71).

In preferred embodiments, the subject for treatment or prevention, according to the present invention, can be a human, or a non-human experimental animal, e.g. a mouse or a rat, a domestic animal, or a non-human primate. The experimental animal can be an animal model for a neurodegenerative disorder, e.g. a transgenic mouse and/or a knock-out mouse with an AD-type neuropathology.

In a further aspect, the invention features a modulator of an activity, or a level, or both said activity and said level of at least one substance which is selected from the group consisting of (i) the gene coding for KCNJ6 protein, and/or (ii) a transcription product of the gene coding for KCNJ6 protein, and/or (iii) a translation product of the gene coding for KCNJ6 protein, and/or (iv) a fragment, or derivative, or variant of (i) to (iii).

In an additional aspect, the invention features a pharmaceutical composition comprising said modulator and preferably a pharmaceutical carrier. Said carrier refers to a diluent, adjuvant, excipient, or vehicle with which the modulator is administered.

In another aspect, the invention provides for the use of a modulator of an activity, or a level, or both said activity and said level of at least one substance which is selected from the group consisting of (i) the gene coding for KCNJ6 protein, and/or (ii) a transcription product of the gene coding for KCNJ6 protein, and/or (iii) a translation product of the gene coding for KCNJ6 protein, and/or (iv) a fragment, or derivative, or variant of (i) to (iii) for a preparation of a medicament for treating or preventing a neurodegenerative disease, in particular AD.

In one aspect, the present invention also provides a kit comprising one or more containers filled with a therapeutically or prophylactically effective amount of said pharmaceutical composition.

In a further aspect, the invention features a recombinant, non-human animal comprising a non-native KCNJ6 gene sequence, or a fragment, or a derivative, or variant thereof. The generation of said recombinant, non-human animal comprises (i) providing a gene targeting construct containing said gene sequence and a selectable marker sequence, and (ii) introducing said targeting construct into a stem cell of a non-human animal, and (iii) introducing said non-human animal stem cell into a non-human embryo, and (iv) transplanting said embryo into a pseudopregnant non-human animal, and (v) allowing said embryo to develop to term, and (vi) identifying a genetically altered non-human animal whose genome comprises a modification of said gene sequence in both alleles, and (vii) breeding the genetically altered non-human animal of step (vi) to obtain a genetically altered non-human animal whose genome comprises a modification of said endogenous gene, wherein said gene is mis-expressed, or under-expressed, or over-expressed, and wherein said disruption or alteration results in said non-human animal exhibiting a predisposition to developing symptoms of a neurodegenerative disease, in particular AD. Strategies and techniques for the generation and construction of such an animal are known to those of ordinary skill in the art (see e.g. Capecchi, Science 1989, 244: 1288-1292 and Hogan et al., Manipulating the Mouse Embryo: A Laboratory Manual, Cold Spring Harbor Laboratory Press, Cold Spring Harbor, N.Y., 1994 and Jackson and Abbott, Mouse Genetics and Transgenics: A Practical Approach, Oxford University Press, Oxford, England, 1999). It is preferred to make use of such a recombinant non-human animal as an animal model for investigating neurodegenerative diseases, in particular Alzheimer's disease. Such an animal may be useful for screening, testing and validating compounds, agents and modulators in the development of diagnostics and therapeutics to treat neurodegenerative diseases, in particular Alzheimer's disease.

In another aspect, the invention features an assay for screening for a modulator of neurodegenerative diseases, in particular AD, or related diseases and disorders of one or more substances selected from the group consisting of (i) the gene coding for KCNJ6 protein, and/or (ii) a transcription product of the gene coding for KCNJ6 protein, and/or (iii) a translation product of the gene coding for KCNJ6 protein, and/or (iv) a fragment, or derivative, or variant of (i) to (iii). This screening method comprises (a) contacting a cell with a test compound, and (b) measuring the activity, or the level, or both the activity and the level of one or more substances recited in (i) to (iv), and (c) measuring the activity, or the level, or both the activity and the level of said substances in a control cell not contacted with said test compound, and (d) comparing the levels of the substance in the cells of step (b) and (c), wherein an alteration in the activity and/or level of said substances in the contacted cells indicates that the test compound is a modulator of said diseases and disorders.

In one further aspect, the invention features a screening assay for a modulator of neurodegenerative diseases, in particular AD, or related diseases and disorders of one or more substances selected from the group consisting of (i) the gene coding for KCNJ6 protein, and/or (ii) a transcription product of the gene coding for KCNJ6 protein, and/or (iii) a translation product of the gene coding for KCNJ6 protein, and/or (iv) a fragment, or derivative, or variant of (i) to (iii), comprising (a) administering a test compound to a test animal which is predisposed to developing or has already developed symptoms of a neurodegenerative disease or related diseases or disorders, and (b) measuring the activity and/or level of one or more substances recited in (i) to (iv), and (c) measuring the activity and/or level of said substances in a matched control animal which is equally predisposed to developing or has already developed said symptoms of a neurodegenerative disease, and to which animal no such test compound has been administered, and (d) comparing the activity and/or level of the substance in the animals of step (b) and (c), wherein an alteration in the activity and/or level of substances in the test animal indicates that the test compound is a modulator of said diseases and disorders.

In a preferred embodiment, said test animal and/or said control animal is a recombinant, non-human animal which expresses the gene coding for KCNJ6 protein, or a fragment thereof, or a derivative thereof, under the control of a transcriptional regulatory element which is not the native KCNJ6 protein gene transcriptional control regulatory element.

In another embodiment, the present invention provides a method for producing a medicament comprising the steps of (i) identifying a modulator of neurodegenerative diseases by a method of the aforementioned screening assays and (ii) admixing the modulator with a pharmaceutical carrier. However, said modulator may also be identifiable by other types of screening assays.

In another aspect, the present invention provides for an assay for testing a compound, preferably for screening a plurality of compounds, for inhibition of binding between a ligand and KCNJ6 protein, or a fragment, or derivative, or variant thereof. Said screening assay comprises the steps of (i) adding a liquid suspension of said KCNJ6 protein, or a fragment, or derivative, or variant thereof, to a plurality of containers, and (ii) adding a compound or a plurality of compounds to be screened for said inhibition to said plurality of containers, and (iii) adding a detectable, preferably a fluorescently labelled ligand to said containers, and (iv) incubating said KCNJ6 protein, or said fragment, or derivative or variant thereof, and said compound or plurality of compounds, and said detectable, preferably fluorescently labelled ligand, and (v) measuring the amounts of preferably the fluorescence associated with said KCNJ6 protein, or with said fragment, or derivative, or variant thereof, and (vi) determining the degree of inhibition by one or more of said compounds of binding of said ligand to said KCNJ6 protein, or said fragment, or derivative, or variant thereof. It might be preferred to reconstitute said KCNJ6 translation product, or fragment, or derivative, or variant thereof into artificial liposomes to generate the corresponding proteoliposomes to determine the inhibition of binding between a ligand and said KCNJ6 translation product.

Methods of reconstitution of KCNJ6 translation products from detergent into liposomes have been detailed (Schwarz et al., Biochemistry 1999, 38: 9456-9464; Krivosheev and Usanov, Biochemistry-Moscow 1997, 62: 1064-1073). Instead of utilizing a fluorescently labelled ligand, it might in some aspects be preferred to use any other detectable label known to the person skilled in the art, e.g. radioactive labels, and detect it accordingly. Said method may be useful for the identification of novel compounds as well as for evaluating compounds which have been improved or otherwise optimized in their ability to inhibit the binding of a ligand to a gene product of the gene coding for KCNJ6 protein, or a fragment, or derivative, or variant thereof. One example of a fluorescent binding assay, in this case based on the use of carrier particles, is disclosed and described in patent application WO 00/52451. A further example is the competitive assay method as described in patent WO 02/01226. Preferred signal detection methods for screening assays of the instant invention are described in the following patent applications: WO 96/13744, WO 98/16814, WO 98/23942, WO 99/17086, WO 99/34195, WO 00/66985, WO 01/59436, WO 01/59416.

In one further embodiment, the present invention provides a method for producing a medicament comprising the steps of (i) identifying a compound as an inhibitor of binding between a ligand and a gene product of the gene coding for KCNJ6 protein by the aforementioned inhibitory binding assay and (ii) admixing the compound with a pharmaceutical carrier. However, said compound may also be identifiable by other types of screening assays.

In another aspect, the invention features an assay for testing a compound, preferably for screening a plurality of compounds to determine the degree of binding of said compounds to KCNJ6 protein, or to a fragment, or derivative, or variant thereof. Said screening assay comprises (i) adding a liquid suspension of said KCNJ6 protein, or a fragment, or derivative, or variant thereof, to a plurality of containers, and (ii) adding a detectable, preferably a fluorescently labelled compound or a plurality of detectable, preferably fluorescently labelled compounds to be screened for said binding to said plurality of containers, and (iii) incubating said KCNJ6 protein, or said fragment, or derivative, or variant thereof, and said detectable, preferably fluorescently labelled compound or detectable, preferably fluorescently labelled compounds, and (iv) measuring the amounts of preferably the fluorescence associated with said KCNJ6 protein, or with said fragment, or derivative, or variant thereof, and (v) determining the degree of binding by one or more of said compounds to said KCNJ6 protein, or said fragment, or derivative, or variant thereof. In this type of assay it might be preferred to use a fluorescent label. However, any other type of detectable label might also be employed. Also in this type of assay it might be preferred to reconstitute a KCNJ6 translation product or a fragment, or derivative, or variant thereof into artificial liposomes as described in the present invention. Said assay methods may be useful for the identification of novel compounds as well as for evaluating compounds which have been improved or otherwise optimized in their ability to bind to KCNJ6 protein, or a fragment, or derivative, or variant thereof.

In one further embodiment, the present invention provides a method for producing a medicament comprising the steps of (i) identifying a compound as a binder to a gene product of the gene coding for KCNJ6 protein by the aforementioned binding assays and (ii) admixing the compound with a pharmaceutical carrier. However, said compound may also be identifiable by other types of screening assays.

In another embodiment, the present invention provides for a medicament obtainable by any of the methods according to the herein claimed screening assays. In one further embodiment, the instant invention provides for a medicament obtained by any of the methods according to the herein claimed screening assays.

Furthermore, in another aspect the present invention features protein molecules and the use of said protein molecules having SEQ ID NO: 1, said protein molecules of Kir3.2 being translation products of the gene coding for KCNJ6, or fragments, or derivatives, or variants thereof, as diagnostic targets for detecting a neurodegenerative disease, in particular Alzheimer's disease.

The present invention further features protein molecules and the use of said protein molecules having SEQ ID NO: 1, said protein molecules Kir3.2 being translation products of the gene coding for KCNJ6, or fragments, or derivatives, or variants thereof, as screening targets for reagents or compounds preventing, or treating, or ameliorating a neurodegenerative disease, in particular Alzheimer's disease.

The present invention features antibodies which are specifically immunoreactive with an immunogen, wherein said immunogen is a translation product of the gene KCNJ6 coding for Kir3.2 protein, having SEQ ID NO: 1, or fragments, or derivatives, or variants thereof. The immunogen may comprise immunogenic or antigenic epitopes or portions of a translation product of said gene, wherein said immunogenic or antigenic portion of a translation product is a polypeptide, and wherein said polypeptide elicits an antibody response in an animal, and wherein said polypeptide is immunospecifically bound by said antibody. Methods for generating antibodies are well known in the art (see Harlow et al., Antibodies, A Laboratory Manual, Cold Spring Harbor Laboratory Press, Cold Spring Harbor, N.Y., 1988). The term “antibody”, as employed in the present invention, encompasses all forms of antibodies known in the art, such as polyclonal, monoclonal, chimeric, recombinatorial, anti-idiotypic, humanized, or single chain antibodies, as well as fragments thereof (see Dubel and Breitling, Recombinant Antibodies, Wiley-Liss, New York, N.Y., 1999). Antibodies of the present invention are useful, for instance, in a variety of diagnostic and therapeutic methods, based on state-in-the-art techniques (see Harlow and Lane, Using Antibodies: A Laboratory Manual, Cold Spring Harbor Laboratory Press, Cold Spring Harbor, N.Y., 1999 and Edwards R., Immunodiagnostics: A Practical Approach, Oxford University Press, Oxford, England, 1999) such as enzyme-immuno assays (e.g. enzyme-linked immunosorbent assay, ELISA), radioimmuno assays, chemoluminescence-immuno assays, Western-blot, immunoprecipitation and antibody microarrays. These methods involve the detection of translation products of the KCNJ 6 gene, or fragments, or derivatives, or variants thereof.

In a preferred embodiment of the present invention, said antibodies can be used for detecting the pathological state of a cell in a sample obtained from a subject, comprising immunocytochemical staining of said cell with said antibody, wherein an altered degree of staining, or an altered staining pattern in said cell compared to a cell representing a known health status indicates a pathological state of said cell. Preferably, the pathological state relates to a neurodegenerative disease, in particular to AD. Immunocytochemical staining of a cell can be carried out by a number of different experimental methods well known in the art. It might be preferred, however, to apply an automated method for the detection of antibody binding, wherein the determination of the degree of staining of a cell, or the determination of the cellular or subcellular staining pattern of a cell, or the topological distribution of an antigen on the cell surface or among organelles and other subcellular structures within the cell, are carried out according to the method described in U.S. Pat. No. 6,150,173.

Other features and advantages of the invention will be apparent from the following description of figures and examples which are illustrative only and not intended to limit the remainder of the disclosure in any way.

### FIGURES

FIG. 1 discloses the initial identification of the differential expression of the KCNJ6 gene coding for Kir3.2 proteins in a fluorescence differential display screen. The figure shows a clipping of a large preparative fluorescent differential display gel. PCR products from the frontal cortex (F) and the temporal cortex (T) of two healthy control subjects and six AD patients were loaded in duplicate onto a denaturing polyacrylamide gel (from left to right). PCR products were obtained by amplification of the individual cDNAs with the corresponding one-base-anchor oligonucleotide and the specific Cy3 labelled random primers. The arrow indicates the migration position where significant differences in intensity of the signals for a transcription product of the KCNJ6 gene derived from frontal cortex and from the temporal cortex of AD patients as compared to healthy controls exist. The differential expression reflects a down-regulation, a decreased expression of human KCNJ6 gene transcription in the temporal cortex compared to the frontal cortex of AD patients. Comparing the signals derived from temporal cortex and frontal cortex of healthy non-AD control subjects with each other, no difference in signal intensity, i.e. no altered expression level can be detected.

FIG. 2 illustrates the verification of the differential expression of the human KCNJ6 gene in AD brain tissues by quantitative RT-PCR analysis. Quantification of RT-PCR products from RNA samples collected from the frontal cortex (F) and the temporal cortex (T) of AD patients (FIG. 2a) and samples from the frontal cortex (F) and the temporal cortex of healthy, age-matched control individuals (FIG. 2b) was performed by the LightCycler rapid thermal cycling technique. The data were normalized to the combined average values of a set of standard genes which showed no significant differences in their gene expression levels. Said set of standard genes consisted of genes for cyclophilin B, the ribosomal protein S9, the transferrin receptor, GAPDH, and beta-actin. The figures depict the kinetics of amplification by plotting the cycle number against the amount of amplified material as measured by its fluorescence. Note that the amplification kinetics of KCNJ6 cDNA from the frontal and temporal cortices of a normal control individual during the exponential phase of the reaction are juxtaposed (FIG. 2b, arrowheads), whereas in Alzheimer's disease (FIG. 2a, arrowheads) there is a significant separation of the corresponding curves, indicating a differential expression of the gene coding for KCNJ6 in the respective analyzed brain regions, indicating a dysregulation, preferably a downregulation of a transcription product of the human KCNJ6 gene, or a fragment, or derivative, or variant thereof, in the temporal cortex relative to the frontal cortex, or an up-regulation of a transcription product of the human KCNJ6 gene, in the frontal cortex relative to the temporal cortex.

FIG. 3 shows the analysis of absolute mRNA expression of KCNJ6 by comparison of control and AD stages using statistical method of the median at 98%-confidence level. The data were calculated by defining control groups including subjects with either Braak stages 0 to 1, Braak stages 0 to 2, or Braak stages 0 to 3 which are compared with the data calculated for the defined AD patient groups including Braak stages 2 to 6, Braak stages 3 to 6 and Braak stages 4 to 6, respectively. Additionally, three groups including subjects with either Braak stages 0 to 1, Braak stages 2 to 3 and Braak stages 4 to 6, respectively, were compared with each other. A significant difference was detected comparing frontal cortex (F) and inferior temporal cortex (T) of AD patients and of healthy age-matched control persons with each other. Said difference reflects a down-regulation of KCNJ6 in the temporal cortex of AD patients relative to the temporal cortex of control persons and a down-regulation of KCNJ6 in the temporal cortex of AD patients compared to their frontal cortices.

FIG. 4 discloses SEQ ID NO: 1, the amino acid sequence of the human KCNJ6 protein Kir3.2 (Genbank accession number P48051). The full length human Kir3.2 protein comprises 423 amino acids.

FIG. 5 shows SEQ ID NO: 2, the nucleotide sequence of the human KCNJ6 cDNA (Genbank accession number U52153) encoding the Kir3.2 protein, comprising 2598 nucleotides.

FIG. 6 depicts SEQ ID NO: 3, the nucleotide sequence of the 57 bp KCNJ6 cDNA fragment, identified and obtained by differential display and subsequent cloning (sequence in 5′ to 3′ direction).

FIG. 7 shows the nucleotide sequence of SEQ ID NO. 4, the coding sequence (cds) of the human KCNJ6 gene, comprising 1272 nucleotides, harbouring nucleotides 652 to 1923 of SEQ ID NO. 2.

FIG. 8 outlines the sequence alignment of SEQ ID NO: 3 to the nucleotide sequence of KCNJ6 cDNA, SEQ ID NO: 2.

FIG. 9 depicts the sequence alignment of the primers used for KCNJ6 transcription level profiling (primer A, SEQ ID NO: 5 and primer B, SEQ ID NO:6) by quantitative RT-PCR with the corresponding clippings of SEQ ID NO: 2, Kir3.2 cDNA.

FIG. 10 schematically charts the alignment of the KCNJ6 cDNA sequence SEQ ID NO: 2, the identified cDNA fragment sequence SEQ ID NO: 3, both primer sequences used for KCNJ6 transcription level profiling (primer A, SEQ ID NO: 5, primer B, SEQ ID NO: 6) and the coding sequence (cds) of KCNJ6. The sequence positions are indicated on the right side.

FIG. 11 lists KCNJ6 gene expression levels in the temporal cortex relative to the frontal cortex in fifteen AD patients, herein identified by internal reference numbers P010, P011, P012, P014, P016, P017, P019, P038, P040, P041, P042, P046, P047, P048, P049 and twenty-six age-matched control individuals, herein identified by internal reference numbers C005, C008, C011, C012, C014, C025, C026, C027, C028, C029, C030, C031, C032, C033, C034, C035, C036, C037, C038, C039, C041, C042, DE02, DE03, DE05, DE07. For an up-regulation in the temporal cortex, the values shown are calculated according to the formula described herein (see below) and in case of an up-regulation in the frontal cortex the reciprocal values are calculated, respectively. An obvious difference reflecting a down-regulation in the temporal cortex, an up-regulation in the frontal cortex is shown. The bar diagram visualizes individual natural logarithmic values of the temporal to frontal cortex, In(IT/IF), and of the frontal to temporal cortex regulation factors, In(IF/IT), in different Braak stages (0 to 6).

FIG. 12 depicts a Western blot image of total cell protein extracts labeled with polyclonal anti-Kir3.2 antibody (Santa Cruz sc16135, 1:200).

Lanes A and B: total protein extract of H4APPsw cells stably expressing Kir3.2 tagged with a myc-tag (Kir3.2-myc, B) and myc-tagged control H4APPsw cells (A). The arrow indicates a major band at about 45 kDa (lane A), which corresponds to the predicted molecular weight of the Kir3.2 protein.

FIG. 13 shows the immunofluorescence analysis of H4APPsw control cells and H4APPsw cells stably over-expressing the myc-tagged Kir3.2 protein (H4APPsw-Kir3.2 cds-myc). The Kir3.2-myc protein was detected with polyclonal anti-myc antibodies (MBL) and a Cy3-conjugated anti-rabbit antibody (Amersham) (FIGS. 13A and 13B). The cellular nucleus was stained with DAPI (FIGS. 13C and 13D). The overlay analysis indicates that the Kir3.2 cds-myc protein is mainly localized at the plasma membranes, within the endoplasmatic reticulum and vesicles (FIG. 13E) and is over-expressed in more than 90% of the H4APPsw-Kir3.2 cds-myc transduced cells as compared to the H4APPsw control cells (FIG. 13F).

FIG. 14 exemplarily depicts micrographs digitally taken from sections of the inferior temporal gyrus (IT, lower panel) and of the frontal cortex (F) from control donors (Control Braak 1) and from persons with Alzheimer disease (Patients Braak 5). The tissue sections are immunolabeled with affinity-purified rabbit polyclonal anti-Kir3.2 antibodies (Upstate, 06-792, 1:15, signals) (magnification 10×). The data exemplarily shown here clearly indicate that the level of intensity and quantity of Kir3.2 immunoreactivity is decreased in the inferior temporal cortex from patients (Braak stage 5) as compared to the inferior temporal cortex from control persons (Braak 1). The findings show that neuronal Kir3.2 immunoreactivity, representing the level of the KCNJ6 translation product, i.e. Kir3.2 protein, is markedly decreased in the temporal cortex from persons with AD as compared to the temporal cortex of healthy control persons. This AD-associated decrease of Kir3.2 immunoreactivity becomes more prominent with increasing Braak stages, indicating that the course of AD, i.e. the progression of AD pathology, is reflected by a strong decrease in Kir3.2 expression which may either accompany or follow or even preced AD neurodegenerative changes. Temporal cortex (T), Frontal cortex (F); Healthy control person (Control); Alzheimer's patient (Patient).

### EXAMPLE

### (i) Brain Tissue Dissection from Patients with AD

Brain tissues from AD patients and age-matched control subjects were collected, on average, within 6 hours post-mortem and immediately frozen on dry ice. Sample sections from each tissue were fixed in paraformaldehyde for histopathological confirmation of the diagnosis. Brain areas for differential expression analysis were identified and stored at −80° C. until RNA extractions were performed.

### (ii) Isolation of Total mRNA

Total RNA was extracted from post-mortem brain tissue by using the RNeasy kit (Qiagen) according to the manufacturer's protocol. The accurate RNA concentration and the RNA quality were determined with the DNA LabChip system using the Agilent 2100 Bioanalyzer (Agilent Technologies). For additional quality testing of the prepared RNA, i.e. exclusion of partial degradation and testing for DNA contamination, specifically designed intronic GAPDH oligonucleotides and genomic DNA as reference control were utilised to generate a melting curve with the LightCycler technology as described in the supplied protocol by the manufacturer (Roche).

### (iii) cDNA Synthesis and Identification of Differentially Expressed Genes by Fluorescence Differential Display (FDD)

In order to identify changes in gene expression in different tissue, a modified and improved differential display (DD) screening method was employed. The original DD screening method is known to those skilled in the art (Liang and Pardee, Science 1995, 267:1186-7). This technique compares two populations of RNA and provides clones of genes that are expressed in one population but not in the other. Several samples can be analyzed simultaneously and both up- and down-regulated genes can be identified in the same experiment. By adjusting and refining several steps in the DD method as well as modifying technical parameters, e.g. increasing redundancy, evaluating optimized reagents and conditions for reverse transcription of total RNA, optimizing polymerase chain reactions (PCR) and separation of the products thereof, a technique was developed which allows for highly reproducible and sensitive results. The applied and improved DD technique was described in detail by von der Kammer et al. (Nucleic Acids Research 1999, 27: 2211-2218). A set of 64 specifically designed random primers were developed (standard set) to achieve a statistically comprehensive analysis of all possible RNA species. Further, the method was modified to generate a preparative DD slab-gel technique, based on the use of fluorescently labelled primers. In the present invention, RNA populations from carefully selected post-mortem brain tissues (frontal and temporal cortex) of Alzheimer's disease patients and age-matched control subjects were compared.

As starting material for the DD analysis we used total RNA, extracted as described above (ii). Equal amounts of 0.05 μg RNA each were transcribed into cDNA in 20 μl reactions containing 0.5 mM each dNTP, 1 μl Sensiscript Reverse Transcriptase and 1×RT buffer (Qiagen), 10 U RNase inhibitor (Qiagen) and 1 μM of either one-base-anchor oligonucleotides HT₁₁A, HT₁₁G or HT₁₁C (Liang et al., Nucleic Acids Research 1994, 22: 5763-5764; Zhao et al., Biotechniques 1995, 18: 842-850). Reverse transcription was performed for 60 min at 37° C. with a final denaturation step at 93° C. for 5 min. 2 μl of the obtained cDNA each was subjected to a polymerase chain reaction (PCR) employing the corresponding one-base-anchor oligonucleotide (1 μM) along with either one of the Cy3 labelled random DD primers (1 μM), 1× GeneAmp PCR buffer (Applied Biosystems), 1.5 mM MgCl₂ (Applied Biosystems), 2 μM dNTP-Mix (dATP, dGTP, dCTP, dTTP Amersham Pharmacia Biotech), 5% DMSO (Sigma), 1 U AmpliTaq DNA Polymerase (Applied Biosystems) in a 20 μl final volume. PCR conditions were set as follows: one round at 94° C. for 30 sec for denaturing, cooling 1° C./sec down to 40° C., 40° C. for 4 min for low-stringency annealing of primer, heating 1° C./sec up to 72° C., 72° C. for 1 min for extension. This round was followed by 39 high-stringency cycles: 94° C. for 30 sec, cooling 1° C./sec down to 60° C., 60° C. for 2 min, heating 1° C./sec up to 72° C., 72° C. for 1 min. One final step at 72° C. for 5 min was added to the last cycle (PCR cycler: Multi Cycler PTC 200, MJ Research). 8 μ7, DNA loading buffer were added to the 20 μl PCR product preparation, denatured for 5 min and kept on ice until loading onto a gel. 3.5 μl each were separated on 0.4 mm thick, 6% polyacrylamide (Long Ranger)/7 M urea sequencing gels in a slab-gel system (Hitachi Genetic Systems) at 2000 V, 60 W, 30 mA, for 1 h 40 min. Following completion of the electrophoresis, gels were scanned with a FMBIO II fluorescence-scanner (Hitachi Genetic Systems), using the appropriate FMBIO II Analysis 8.0 software. A full-scale picture was printed, differentially expressed bands marked, excised from the gel, transferred into 1.5 ml containers, overlayed with 200 μl sterile water and kept at −20° C. until extraction.

Elution and reamplification of DD products: The differential bands were extracted from the gel by boiling in 200 μl H₂O for 10 min, cooling down on ice and precipitation from the supernatant fluids by using ethanol (Merck) and glycogen/sodium acetate (Merck) at −20° C. over night, and subsequent centrifugation at 13.000 rpm for 25 min at 4° C. Pellets were washed twice in ice-cold ethanol (80%), resuspended in 10 mM Tris pH 8.3 (Merck) and dialysed against 10% glycerol (Merck) for 1 h at room temperature on a 0.025 μm VSWP membrane (Millipore). The obtained preparations were used as templates for reamplification by 15 high-stringency cycles in 25-μl PCR mixtures containing the corresponding primer pairs as used for the DD PCR (see above) under identical conditions, with the exception of the initial round at 94° C. for 5 min, followed by 15 cycles of: 94° C. for 45 sec, 60° C. for 45 sec, ramp 1° C./sec to 70° C. for 45 sec, and one final step at 72° C. for 5 min.

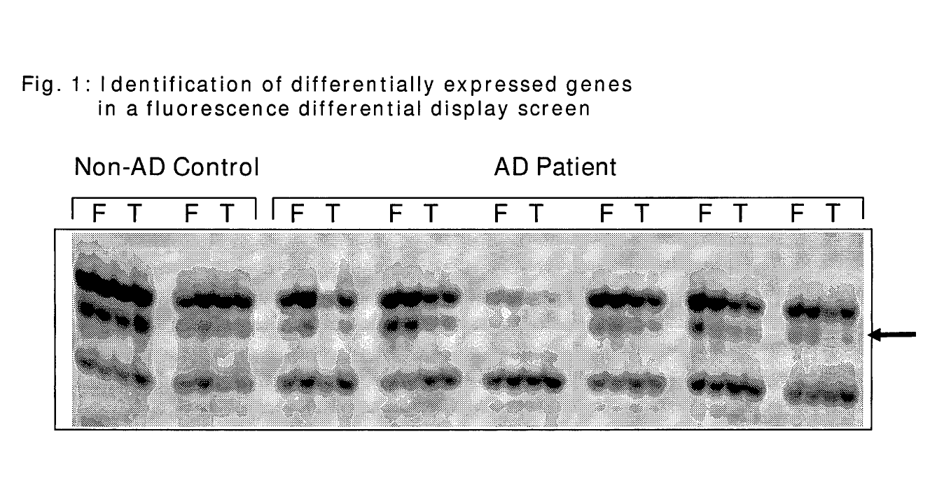

Cloning and sequencing of DD products: Re-amplified cDNAs were analyzed with the DNA LabChip system (Agilent 2100 Bioanalyzer, Agilent Technologies) and ligated into the pCR-Blunt II-TOPO vector and transformed into E. coli Top10F′ cells (Zero Blunt TOPO PCR Cloning Kit, Invitrogen) according to the manufacturer's instructions. Cloned cDNA fragments were sequenced by commercially available sequencing facilities. The result of one such FDD experiment for the gene coding for KCNJ6 protein is shown in FIG. 1.

### (iv) Confirmation of Differential Expression by Quantitative RT-PCR

Positive corroboration of differential KCNJ6 gene expression was performed using the LightCycler technology (Roche). This technique features rapid thermal cyling for the polymerase chain reaction as well as real-time measurement of fluorescent signals during amplification and therefore allows for highly accurate quantification of RT-PCR products by using a kinetic, rather than endpoint readout. The ratios of KCNJ6 cDNAs from the temporal cortices of AD patients and of healthy age-matched control individuals, from the frontal cortices of AD patients and of healthy age-matched control individuals, and the ratios of KCNJ6 cDNAs from the temporal cortex and frontal cortex of AD patients and of healthy age-matched control individuals, respectively, were determined (relative quantification).

The mRNA expression profiling between frontal cortex tissue (F) and inferior temporal cortex tissue (T) of KCNJ6 has been analyzed in four up to nine tissues per Braak stage. Because of the lack of high quality tissues from one donor with Braak 3 pathology, tissues of one additional donor with Braak 2 pathology were included, and because of the lack of high quality tissues from one donor with Braak 6 pathology, tissue samples of one additional donor with Braak 5 pathology were included.

For the analysis of the profiling, two general approaches have been applied. Both comparative profiling studies, frontal cortex against inferior temporal cortex as well as control against AD patients, which contribute to the complex view of the relevance of KCNJ6 in AD physiology, are shown in detail below.

1) Relative Comparison of the mRNA Expression Between Frontal Cortex Tissue and Inferior Temporal Cortex Tissue of Controls and of Ad Patients, Respectively.

This approach allowed to verify that the identified gene KCNJ6 is either involved in the protection of the less vulnerable tissue (frontal cortex) against degeneration, or is involved in or enhances the process of degeneration in the more vulnerable tissue (inferior temporal cortex).

First, a standard curve was generated to determine the efficiency of the PCR with specific primers for the gene coding for KCNJ6:

Primer A, SEQ ID NO: 5, 5′-CATTTGTGGCCCAAGCCT-3′ (nucleotides 2148-2165 of SEQ ID NO: 2) and Primer B, SEQ ID NO: 6, 3′-ACCTGGGATATGACAAGCAAGG-5′ (nucleotides 2271-2292 of SEQ ID NO: 2). PCR amplification (95° C. and 1 sec, 56° C. and 5 sec, and 72° C. and 5 sec) was performed in a volume of 20 μl containing LightCycler-FastStart DNA Master SYBR Green I mix (contains FastStart Taq DNA polymerase, reaction buffer, dNTP mix with dUTP instead of dTTP, SYBR Green I dye, and 1 mM MgCl₂; Roche), 0.5 μM primers, 2 μl of a cDNA dilution series (final concentration of 40, 20, 10, 5, 1 and 0.5 ng human total brain cDNA; Clontech) and, depending on the primers used, additional 3 mM MgCl₂. Melting curve analysis revealed a single peak at approximately 81.5° C. with no visible primer dimers. Quality and size of the PCR product were determined with the DNA LabChip system (Agilent 2100 Bioanalyzer, Agilent Technologies). A single peak at the expected size of 145 bp for the gene coding for KCNJ6 protein was observed in the electropherogram of the sample.

In an analogous manner, the PCR protocol was applied to determine the PCR efficiency of a set of reference genes which were selected as a reference standard for quantification. In the present invention, the mean value of five such reference genes was determined: (1) cyclophilin B, using the specific primers SEQ ID NO:7, 5′-ACTGAAGCACTACGGGCCTG-3′ and SEQ ID NO:8, 5′-AGCCGTTGGTGTCTTTGCC-3′ except for MgCl₂ (an additional 1 mM was added instead of 3 mM). Melting curve analysis revealed a single peak at approximately 87° C. with no visible primer dimers. Agarose gel analysis of the PCR product showed one single band of the expected size (62 bp). (2) Ribosomal protein S9 (RPS9), using the specific primers SEQ ID NO:9, 5′-GGTCAAATTTACCCTGGCCA-3′ and SEQ ID NO:10, 5′-TCTCATCAAGCGTCAGCAGTTC-3′ (exception: additional 1 mM MgCl₂ was added instead of 3 mM). Melting curve analysis revealed a single peak at approximately 85° C. with no visible primer dimers. Agarose gel analysis of the PCR product showed one single band with the expected size (62 bp). (3) beta-actin, using the specific primers SEQ ID NO:11, 5′-TGGAACGGTGAAGGTGACA-3′ and SEQ ID NO:12, 5′-GGCAAGGGACTTCCTGTAA-3′. Melting curve analysis revealed a single peak at approximately 87° C. with no visible primer dimers. Agarose gel analysis of the PCR product showed one single band with the expected size (142 bp). (4) GAPDH, using the specific primers SEQ ID NO:13, 5′-CGTCATGGGTGTGAACCATG-3′ and SEQ ID NO:14, 5′-GCTAAGCAGTTGGTGGTGCAG-3′. Melting curve analysis revealed a single peak at approximately 83° C. with no visible primer dimers. Agarose gel analysis of the PCR product showed one single band with the expected size (81 bp). (5) Transferrin receptor TRR, using the specific primers SEQ ID NO:15, 5′-GTCGCTGGTCAGTTCGTGATT-3′ and SEQ ID NO:16, 5′-AGCAGTTGGCTGTTGTACCTCTC-3′. Melting curve analysis revealed a single peak at approximately 83° C. with no visible primer dimers. Agarose gel analysis of the PCR product showed one single band with the expected size (80 bp).

For calculation of the values, first the logarithm of the cDNA concentration was plotted against the threshold cycle number Cₜ for the gene coding for KCNJ6 protein and the five reference standard genes. The slopes and the intercepts of the standard curves (i.e. linear regressions) were calculated for all genes. In a second step, cDNAs from frontal cortices of AD patients and of healthy control individuals, from temporal cortices of AD patients and of healthy control individuals, and cDNAs from the frontal cortex and the temporal cortex of AD patients and of control individuals, respectively, were analyzed in parallel and normalized to cyclophilin B. The Cₜ values were measured and converted to ng total brain cDNA using the corresponding standard curves:
10ˆ(Cₜ value−intercept)/slope [ng total brain cDNA]

The values for temporal and frontal cortex KCNJ6 cDNAs, and the values from the frontal cortex KCNJ6 cDNAs of AD patients (P) and control individuals (C), and the values for temporal cortex KCNJ6 cDNAs of AD patients (P) and of control individuals (C), respectively, were normalized to cyclophilin B and the ratios were calculated according to formulas:

$$Ratio = \frac{KCNJ 6 temporal [ ng ] / cyclophilin B temporal [ ng ]}{KCNJ 6 frontal [ ng ] / cyclophilin B frontal [ ng ]}$$

$$Ratio = \frac{KCNJ 6 P temporal [ ng ] / cyclophilin B P temporal [ ng ]}{KCNJ 6 C temporal [ ng ] / cyclophilin B C frontal [ ng ]}$$

$$Ratio = \frac{KCNJ 6 P frontal [ ng ] / cyclophilin B P frontal [ ng ]}{KCNJ 6 C frontal [ ng ] / cyclophilin B C frontal [ ng ]}$$

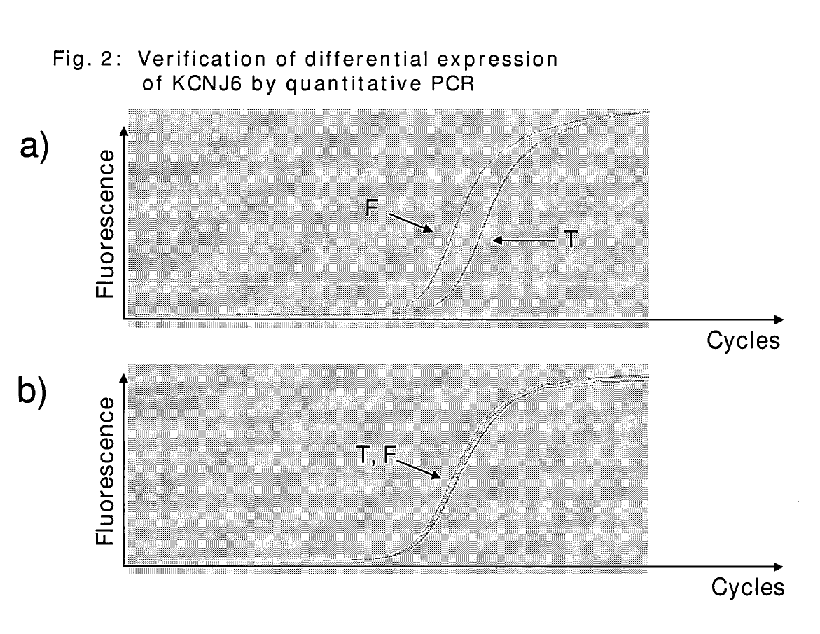

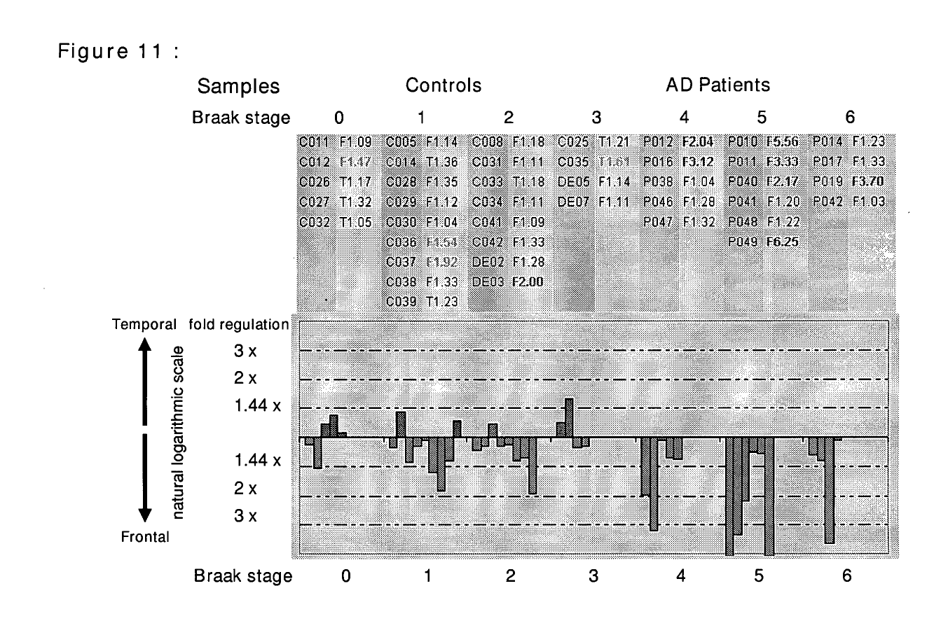

In a third step, the set of reference standard genes was analyzed in parallel to determine the mean average value of the AD patient to control person temporal cortex ratios, of the AD patient to control person frontal cortex ratios, and of the temporal to frontal ratios of AD patients and control persons, respectively, of expression levels of the reference standard genes for each individual brain sample. As cyclophilin B was analyzed in step 2 and step 3, and the ratio from one gene to another gene remained constant in different runs, it was possible to normalize the values for the gene coding for KCNJ6 protein to the mean average value of the set of reference standard genes instead of normalizing to one single gene alone. The calculation was performed by dividing the respective ratio shown above by the deviation of cyclophilin B from the mean value of all housekeeping genes. The results of such quantitative RT-PCR analysis for the gene coding for KCNJ6 protein are shown in FIGS. 2, 3, 11.

2) Comparison of the mRNA Expression Between Controls and AD Patients.

For this analysis it was proven that absolute values of real-time quantitative PCR (Lightcycler method) between different experiments at different time points are consistent enough to be used for quantitive comparisons without usage of calibrators. Cyclophilin was used as a standard for normalization in any of ENS qPCR experiments for more than 100 tissues. Between others it was found to be the most consistently expressed housekeeping gene in our normalization experiments. Therefore a proof of concept was done by using values that were generated for cyclophilin.

First analysis used cyclophilin values from qPCR experiments of frontal cortex and inferior temporal cortex tissues from three different donors. From each tissue the same cDNA preparation was used in all analyzed experiments. Within this analysis no normal distribution of values was achieved due to small number of data. Therefore the method of median and its 98%-confidence level was applied. This analysis revealed a middle deviation of 8.7% from the median for comparison of absolute values and a middle deviation of 6.6% from the median for relative comparison.

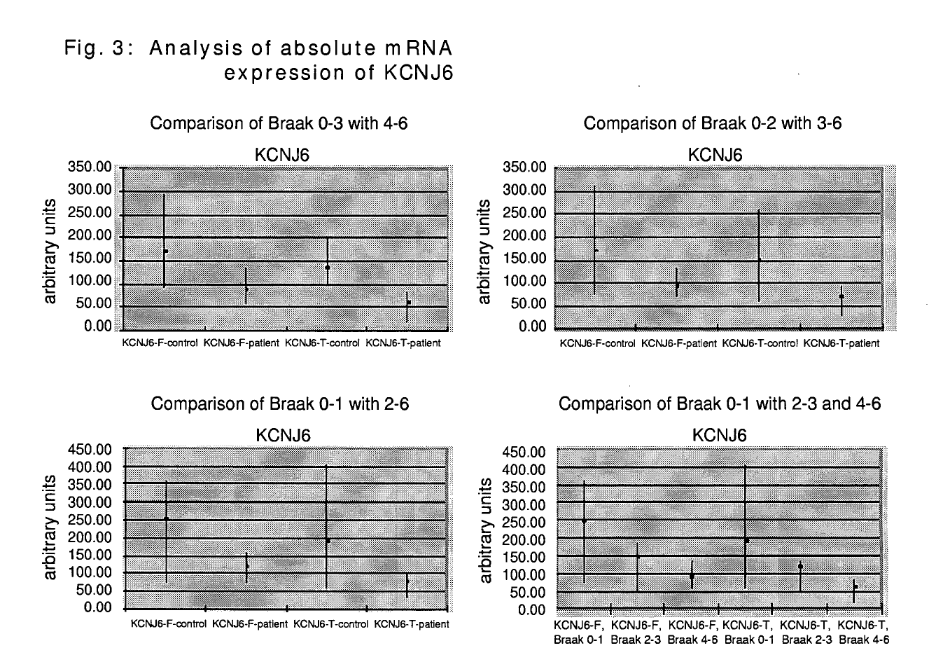

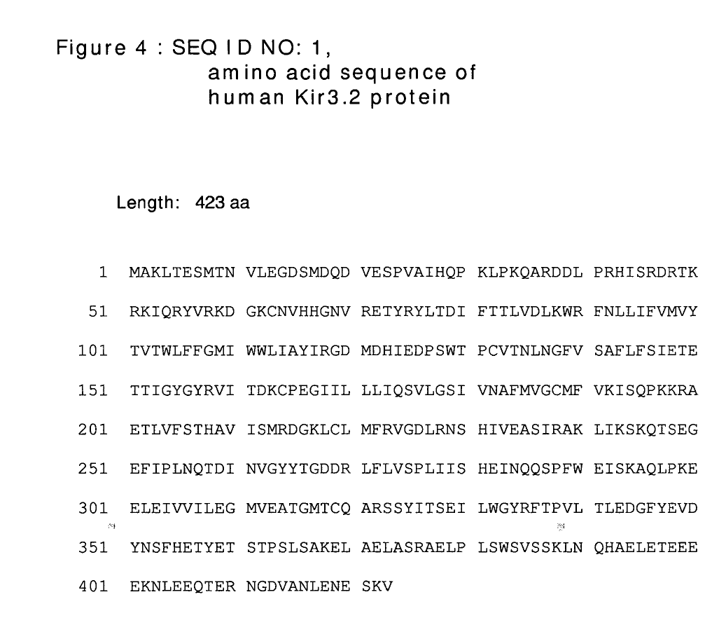

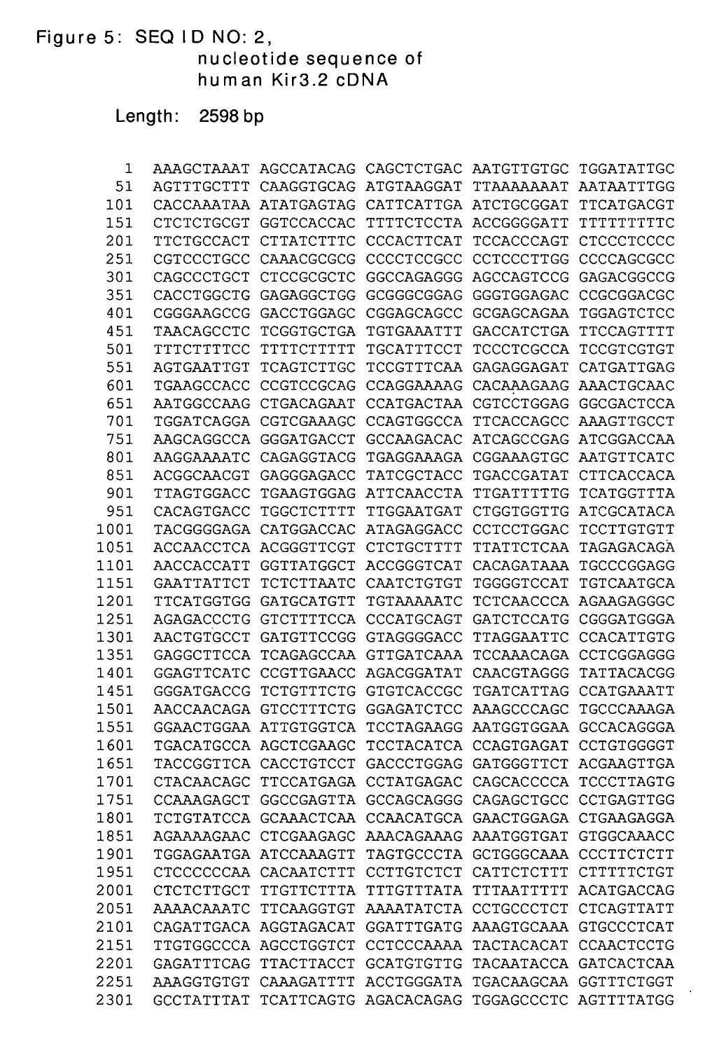

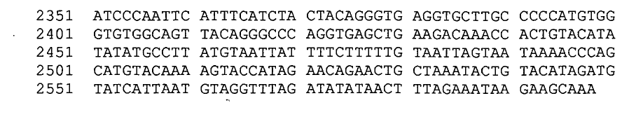

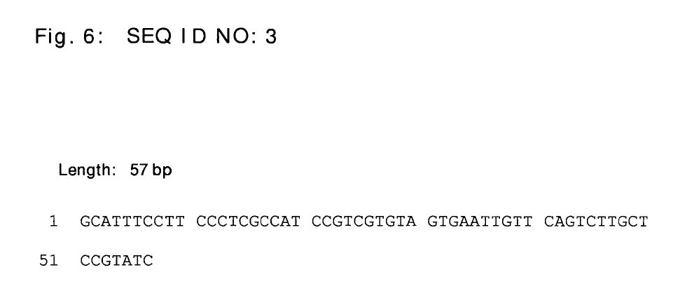

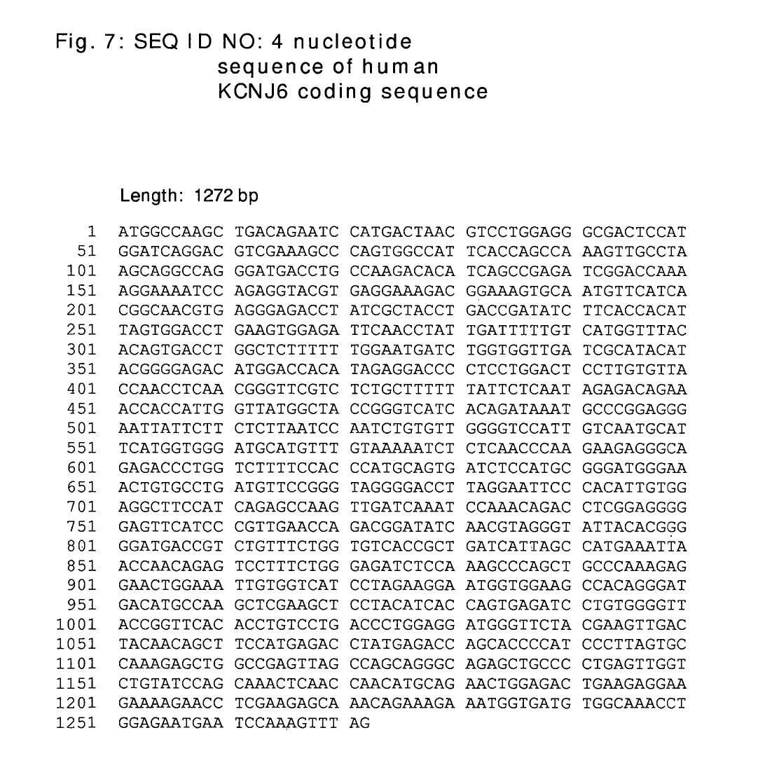

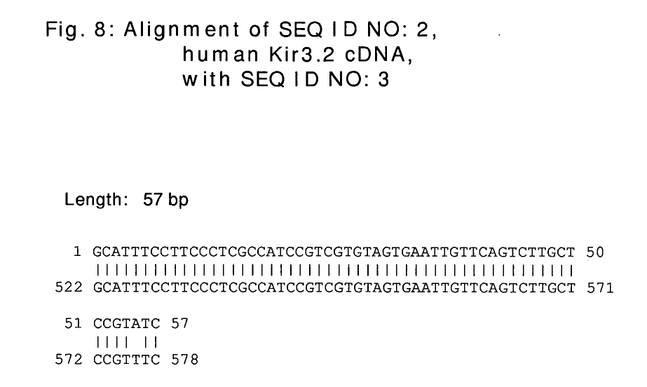

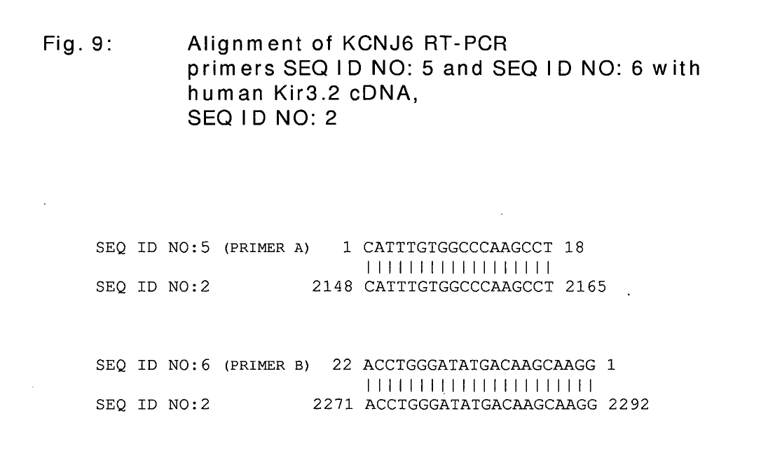

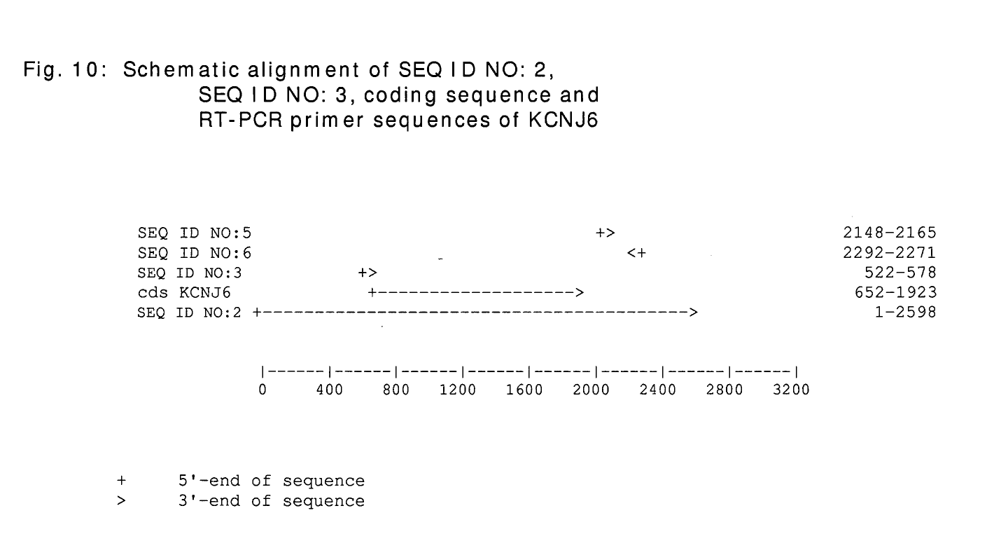

Second analysis used cyclophilin values from qPCR experiments of frontal cortex and inferior temporal cortex tissues from two different donors each, but different cDNA preparations from different time points were used. This analysis revealed a middle deviation of 29.2% from the median for comparison of absolute values and a middle deviation of 17.6% from the median for relative comparison. From this analysis it was concluded, that absolute values from qPCR experiments can be used, but the middle deviation from median should be taken into further considerations. A detailed analysis of absolute values for KCNJ6 was performed. Therefore, absolute levels of KCNJ6 were used after relative normalization with cyclophilin. The median as well as the 98%-confidence level was calculated for the control group (Braak 0-Braak 3) and the patient group (Braak 4-Braak 6), respectively. Same analysis was done redefining the control group (Braak 0-Braak 2) and the patient group (Braak 3-Braak 6) as well as redefining the control group (Braak 0-Braak 1) and the patient group (Braak 2-Braak 6). The latter analysis was aimed to identify early onset of mRNA expression differences between controls and AD patients. In another view of this analysis, three groups comprising Braak stages 0-1, Braak stages 2-3, and Braak stages 4-6, respectively, were compared to each other in order to identify tendencies of gene expression regulation as well as early onset differences. Said analysis described above is shown in FIG. 3.

### (v) Immunoblotting

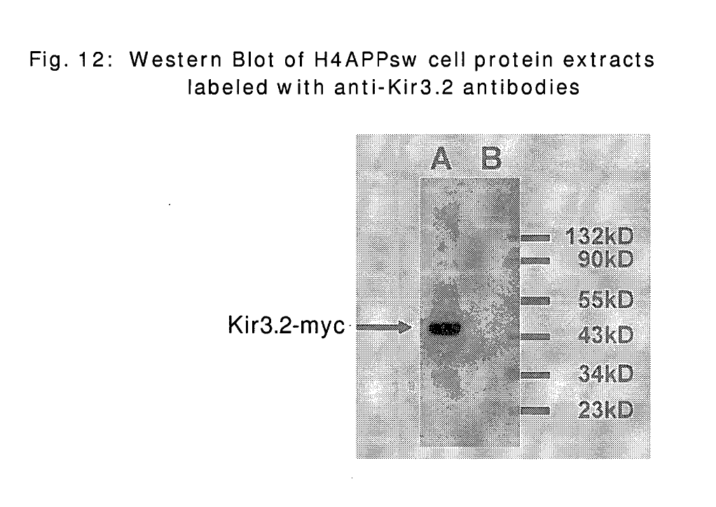

Total protein extract was obtained from H4APPsw cells stably expressing Kir3.2-myc by homogenization in 1 ml RIPA buffer (150 mM sodium chloride, 50 mM tris-HCl, pH7.4, 1 mM ethylenediamine-tetraacetic acid, 1 mM phenylmethylsulfonyl flouride, 1% Triton X-100, 1% sodium deoxycholic acid, 1% sodium dodecylsulfate, 5 μg/ml of aprotinin, 5 μg/ml of leupeptin) on ice. After centrifuging twice for 5 min at 3000 rpm at 4° C., the supernatant was diluted five-fold in SDS-loading buffer. Aliquots of 12 μl of the diluted sample were resolved by SDS-PAGE (8% polyacrylamide) and transferred to PVDF Western Blotting membranes (Boehringer Mannheim). The blots were probed with rabbit polyclonal anti-Kir3.2 antibody (Santa Cruz sc-16135, 1:200) followed by horseradish peroxidase-coupled goat anti-rabbit IgG antiserum (Santa Cruz sc-2030, diluted 1:5000) and developed with the ECL chemoluminescence detection kit (Amersham Pharmacia) (FIG. 12).

### (vi) Immunofluorescence Analysis

For the immunofluorescence staining of Kir3.2 protein in cells, a human neuroglioma cell line was used (H4 cells) which stably expresses the human APP695 isoform carrying the Swedish mutation (K670N, M671L) (H4APPsw cells). The H4APPsw cells were transduced with a pFB-Neo vector (Stratagene, #217561) containing the coding sequence of Kir3.2 (Kir3.2 cds, 1272 bp, SEQ ID NO: 4) and a myc-tag (pFB-Neo-CMV-Kir3.2 cds-myc, Kir3.2-myc vector, 8542 bp) under the control of a strong CMV promotor. For the generation of the Kir3.2-myc vector, the Kir3.2 cds-myc sequence was introduced into the multiple cloning site (MCS) of the pFB-Neo vector. For transduction of the H4APPsw cells with the Kir3.2-myc vector the retroviral expression system ViraPort from Stratagene was used.

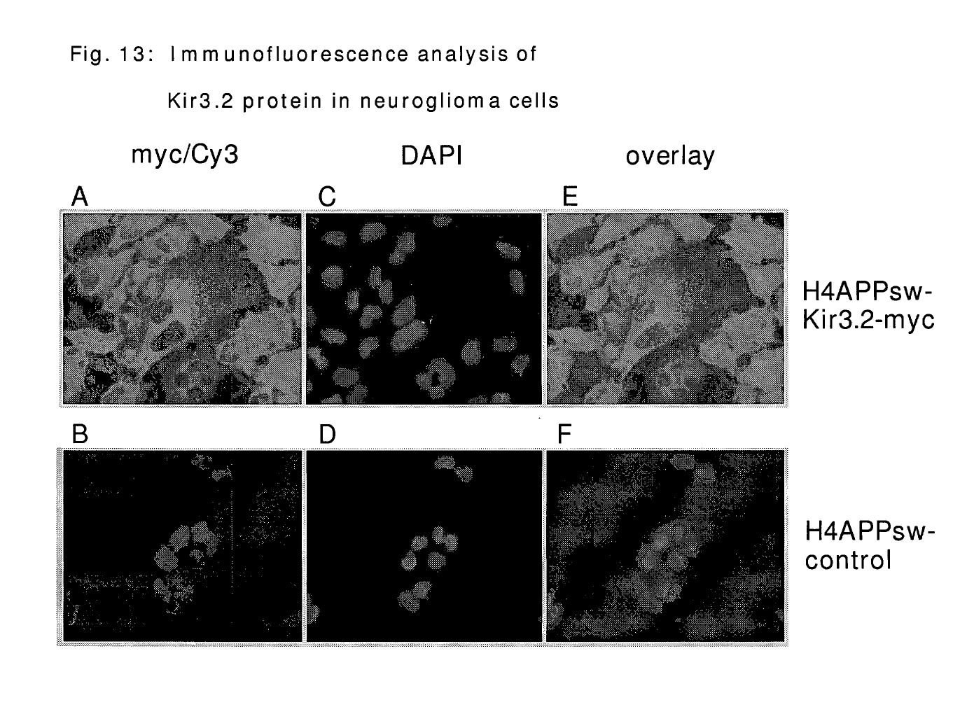

The myc-tagged Kir3.2 over-expressing cells (H4APPsw-Kir3.2-myc) were seeded onto glass cover slips in a 24 well plate (Nunc, Roskilde, Denmark; #143982) at a density of 5×10⁴ cells and incubated at 37° C. at 5% CO₂ over night. To fix the cells onto the cover slip, medium was removed and chilled methanol (−20° C.) was added. After an incubation period of 15 minutes at −20° C., methanol was removed and the fixed cells were blocked for 1 hour in blocking solution (200 μl PBS/5% BSA/3% goat serum) at room temperature. The first antibody (polyclonal anti-myc antibody, rabbit, 1:5000, MBL) and DAPI (DNA-stain, 0.05 μg/ml, 1:1000) in PBS/1% goat serum was added and incubated for 1 hour at room temperature. After removing the first antibody, the fixed cells were washed 3 times with PBS for 5 minutes. The second antibody (Cy3-conjugated anti-rabbit antibody, 1:1000, Amersham Pharmacia, Germany) was applied in blocking solution and incubated for 1 hour at room temperature. The cells were washed 3 times in PBS for 5 minutes. Coverslips were mounted onto microscope slides using Permafluor (Beckman Coulter) and stored over night at 4° C. to harden the mounting media. Cells were visualized using microscopic dark field epifluorescence and bright field phase contrast illumination conditions (IX81, Olympus Optical). Microscopic images (FIG. 13) were digitally captured with a PCO SensiCam and analysed using the appropriate software (AnalySiS, Olympus Optical).

### (vii) Immunohistochemistry

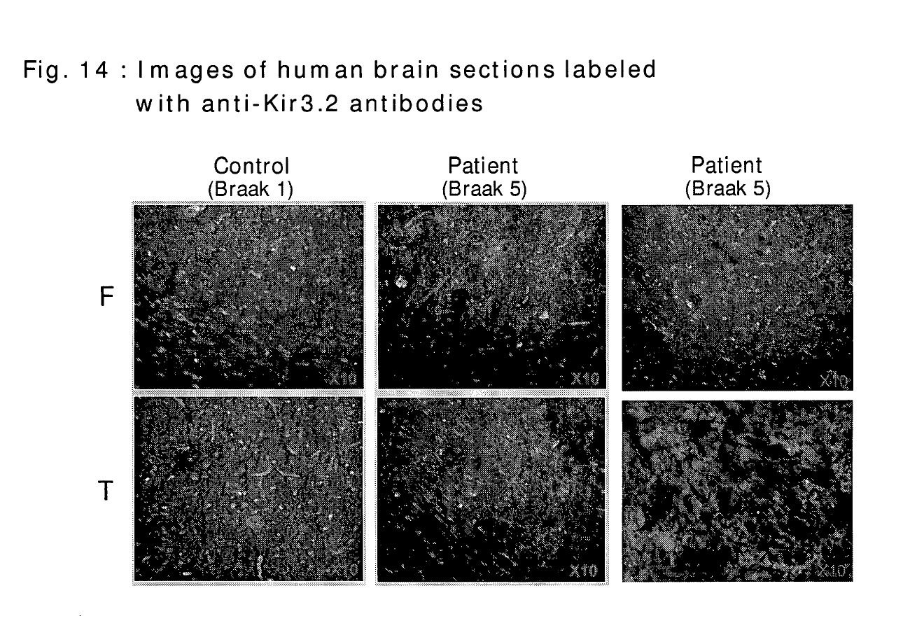

For immunofluorescence staining of KCNJ6, respectively Kir3.2, in human brain, and for the comparison of AD-affected tissue with control tissues, post-mortem fresh-frozen frontal and temporal forebrain specimens from donors comprising patients with clinically diagnosed and neuropathologically confirmed Alzheimer's disease at various Braak stages (Braak 5 exemplarily shown in FIG. 14), as well as age-matched control individuals without Alzheimer (Braak 1 exemplarily shown in FIG. 14), were cut at 14 μm thickness using a cryostat (Leica CM3050S). The tissue sections were air-dried and fixed in ice-chilled acetone for 20 min, or in 4% PFA for 10 min at room temperature. After washing in PBS, the sections were pre-incubated with blocking buffer (10% normal goat serum, 0.2% Triton X-100 in PBS) for 30 min and then incubated with affinity-purified rabbit polyclonal anti-Kir3.2 antibody (1:15 diluted in blocking buffer; Upstate 06-792; amino acids 18-49) overnight at 4° C. After rinsing three times in 0.1% Triton X-100/PBS, the sections were incubated with FITC-conjugated goat anti-rabbit IgG antiserum (Jackson/Dianova, No. 111-096-045, 1:150 diluted in 1% BSA/PBS) for 2 hours at room temperature and then again washed in PBS. Staining of the nuclei was performed by incubation of the sections with 5 μM DAPI in PBS for 3 min. Staining of the neuronal cells was performed by using a mouse monoclonal antibody against the neuronal specific marker NeuN (Chemicon, MAB377, dilution 1:400) and a secondary Cy3-conjugated goat anti-mouse antibody (Dianova, 115-166-062, dilution 1:600) (Neuronal immunoreactivity shown in FIG. 14). Staining of astrocytes was performed by using an antibody against the astrocyte-specific marker GFAP (Abcam, AB7806, dilution 1:300), staining of microglia was performed by using an antibody against the microglial specific marker CD68 (DAKO, M0718, dilution 1:200) and staining against oligodendrocytes by using an antibody against the oligodendrocyte specific marker CNPase (Sigma, C5922, dilution 1:400). In general, immunoreactivity of Kir3.2 was mainly observed in the cerebral cortex, in the neuronal somata as well as in the neuropil and in some proximal dendrites. Kir3.2 immunoreactivity is virtually not detected in astrocytes, CD68-positive microglia, CNPase-positive oligodendrocytes, and it is not associated with myelin. In order to block the autofluoresence of lipofuscin in human brain, the sections were treated with 1% Sudan Black B in 70% ethanol for 2-10 min at room temperature and then sequentially dipped in 70% ethanol, distilled water and PBS. The sections were coverslipped with ‘Vectashield’ mounting medium (Vector Laboratories, Burlingame, Calif.). Microscopic images were obtained using dark field epifluorescence and bright field phase contrast illumination conditions (IX81, Olympus Optical). Microscopic images were digitally captured with a PCO SensiCam and analyzed using the appropriate software (AnalySIS, Olympus Optical) (see FIGS. 13, 14).

1: A method of diagnosing or prognosticating Alzheimer's disease in a subject, or determining whether a subject is at increased risk of developing said disease, comprising determining a level and/or an activity of one or more of a member of the group consisting of:
(i) a transcription product of the gene coding for KCNJ6 proteins,
(ii) a translation product of the gene coding for KCNJ6 proteins, and
(iii) a fragment, or derivative, or variant of said transcription or translation product, in a sample obtained from said subject and comparing said level and/or said activity of said transcription product and/or said translation product to a reference value representing a known disease status and/or to a reference value representing a known health status, and said level and/or said activity is varied compared to a reference value representing a known health status, and/or is similar or equal to a reference value representing a known disease status, thereby diagnosing or prognosticating Alzheimer's disease in said subject, or determining whether said subject is at increased risk of developing said disease.

2: A kit for diagnosing or prognosticating a neurodegenerative disease, in particular Alzheimer's disease, in a subject, or determining the propensity or predisposition of a subject to develop such a disease, said kit comprising at least one reagent which is selected from the group consisting of (i) reagents that detect a transcription product of the gene coding for KCNJ6 proteins having SEQ ID NO:1 and/or fragments, or derivatives, or variants thereof and/or (ii) reagents that detect a translation product of the gene coding for KCNJ6 proteins having SEQ ID NO:1 and/or fragments, or derivatives, or variants thereof.

3: A modulator of an activity and/or of a level of at least one substance which is selected from the group consisting of:
(i) a gene coding for KCNJ6 proteins having SEQ ID NO:1,
(ii) a transcription product of the gene coding for KCNJ6 proteins having SEQ ID NO:1,
(iii) a translation product of the gene coding for KCNJ6 proteins having SEQ ID NO:1, and
(iv) a fragment, or derivative, or variant of (i) to (iii).

4: A recombinant, non-human animal comprising a non-native gene sequence coding for a KCNJ6 protein having SEQ ID NO:1, or a fragment, or derivative, or variant thereof, said animal being obtainable by:
(i) providing a gene targeting construct comprising said gene sequence and a selectable marker sequence,
(ii) introducing said targeting construct into a stem cell of a non-human animal,
(iii) introducing said non-human animal stem cell into a non-human embryo,
(iv) transplanting said embryo into a pseudopregnant non-human animal,
(v) allowing said embryo to develop to term,
(vi) identifying a genetically altered non-human animal whose genome comprises a modification of said gene sequence in both alleles, and
(vii) breeding the genetically altered non-human animal of step (vi) to obtain a genetically altered non-human animal whose genome comprises a modification of said endogenous gene, wherein said disruption results in said non-human animal exhibiting a predisposition to developing symptoms of a neurodegenerative disease or related diseases or disorders.

5: A method for screening, testing, and validating compounds, agents, and modulators in the development of diagnostics and therapeutics to treat neurodegenerative diseases, in particular Alzheimer's disease, the method comprising use of the recombinant, non-human animal according to claim 4.

6: An assay for screening for the modulator of claim 3, comprising:
(a) contacting a cell with a test compound;
(b) measuring the activity and/or level of one or more substances recited in (i) to (iv);
(c) measuring the activity and/or level of one or more substances recited in (i) to (iv) in a control cell not contacted with said test compound; and comparing the levels and/or activities of the substance in the cells of step (b) and (c), wherein an alteration in the activity and/or level of substances in the contacted cells indicates that the test compound is a modulator of said diseases or disorders.

7: A method of screening for the a modulator of claim 3 comprising:
(a) administering a test compound to a non-human test animal which is predisposed to developing or has already developed symptoms of a neurodegenerative disease or related diseases or disorders in respect of the substances recited in (i) to (iv);
(b) measuring the activity and/or level of one or more substances recited in (i) to (iv);
(c) measuring the activity and/or level of one or more substances recited in (i) or (iv) in a matched non-human control animal which is predisposed to developing or has already developed symptoms of a neurodegenerative disease or related diseases or disorders in respect to the substances recited in (i) to (iv) and to which non-human animal no such test compound has been administered;
(d) comparing the activity and/or level of the substance in the animals of step (b) and (c), wherein an alteration in the activity and/or level of substances in the non-human test animal indicates that the test compound is a modulator of said diseases or disorders.

8: The method according to claim 7 wherein said non-human test animal and/or said non-human control animal is a recombinant animal which expresses KCNJ6 having SEQ ID NO:1, or a fragment, or derivative, or variant thereof, under the control of a transcriptional control element which is not the native KCNJ6 gene transcriptional control element.

9: An assay for testing a compound, preferably for screening a plurality of compounds to determine the degree of binding of said compounds to a KCNJ6 protein having SEQ ID NO:1, or to a fragment, or derivative, or variant thereof, said assay comprising the steps of:
(i) adding a liquid suspension of said KCNJ6 protein, or a fragment, or derivative, or variant thereof, to a plurality of containers;
(ii) adding a detectable, in particular a fluorescently labelled compound or a plurality of detectable, in particular fluorescently labelled compounds to be screened for said binding to said plurality of containers;
(iii) incubating said KCNJ6 protein, or said fragment, or derivative, or variant thereof, and said detectable, in particular fluorescently labelled compound or detectable, in particular fluorescently labelled compounds;
(iv) measuring amounts of preferably the fluorescence associated with said KCNJ6 protein, or with said fragment, or derivative, or variant thereof; and
(v) determine the degree of binding by one or more of said compounds to said KCNJ6 protein, or said fragment, or derivative, or variant thereof.

10-12. (canceled)


SEQUENCE LISTING (16 sequences)

SEQ ID NO: 1 - DNA, 18 bp, Artificial Sequence
  Description of Artificial Sequenceprimer for the human KCNJ6 gene
catttgtggcccaagcct

SEQ ID NO: 2 - DNA, 22 bp, Artificial Sequence
  Description of Artificial Sequenceprimer for the human KCNJ6 gene
acctgggatatgacaagcaagg

SEQ ID NO: 3 - DNA, 20 bp, Artificial Sequence
  Description of Artificial Sequenceprimer for the human cyclophilin B gene
actgaagcactacgggcctg

SEQ ID NO: 4 - DNA, 19 bp, Artificial Sequence
  Description of Artificial Sequenceprimer for the human cyclophilin B gene
agccgttggtgtctttgcc

SEQ ID NO: 5 - DNA, 20 bp, Artificial Sequence
  Description of Artificial Sequenceprimer for the human ribosomal protein S9 gene
ggtcaaatttaccctggcca

SEQ ID NO: 6 - DNA, 22 bp, Artificial Sequence
  Description of Artificial Sequenceprimer for the human ribosomal protein S9 gene
tctcatcaagcgtcagcagttc

SEQ ID NO: 7 - DNA, 19 bp, Artificial Sequence
  Description of Artificial Sequenceprimer for the human beta-actin gene
tggaacggtgaaggtgaca

SEQ ID NO: 8 - DNA, 19 bp, Artificial Sequence
  Description of Artificial Sequenceprimer for the human beta-actin gene
ggcaagggacttcctgtaa

SEQ ID NO: 9 - DNA, 20 bp, Artificial Sequence
  Description of Artificial Sequenceprimer for the human GAPDH gene
cgtcatgggtgtgaaccatg

SEQ ID NO: 10 - DNA, 21 bp, Artificial Sequence
  Description of Artificial Sequenceprimer for the human GAPDH gene
gctaagcagttggtggtgcag

SEQ ID NO: 11 - DNA, 21 bp, Artificial Sequence
  Description of Artificial Sequenceprimer for the human transferrin receptor TRR gene
gtcgctggtcagttcgtgatt

SEQ ID NO: 12 - DNA, 23 bp, Artificial Sequence
  Description of Artificial Sequenceprimer for the human transferrin receptor TRR gene
agcagttggctgttgtacctctc

SEQ ID NO: 13 - protein, 423 aa, Homo sapiens
MAKLTESMTNVLEGDSMDQDVESPVAIHQPKLPKQARDDLPRHISRDRTKRKIQRYVRKDGKCNVHHGNVRETYRYLTDIFTTLVDLKWRFNLLIFVMVYTVTWLFFGMIWWLIAYIRGDMDHIEDPSWTPCVTNLNGFVSAFLFSIETETTIGYGYRVITDKCPEGIILLLIQSVLGSIVNAFMVGCMFVKISQPKKRAETLVFSTHAVISMRDGKLCLMFRVGDLRNSHIVEASIRAKLIKSKQTSEGEFIPLNQTDINVGYYTGDDRLFLVSPLIISHEINQQSPFWEISKAQLPKEELEIVVILEGMVEATGMTCQARSSYITSEILWGYRFTPVLTLEDGFYEVDYNSFHETYETSTPSLSAKELAELASRAELPLSWSVSSKLNQHAELETEEEEKNLEEQTERNGDVANLENESKV

SEQ ID NO: 14 - DNA, 2598 bp, Artificial Sequence
  Description of Artificial Sequencenucleotide sequence of the human KCNJ6 cDNA
aaagctaaatagccatacagcagctctgacaatgttgtgctggatattgcagtttgctttcaaggtgcagatgtaaggatttaaaaaaataataatttggcaccaaataaatatgagtagcattcattgaatctgcggatttcatgacgtctctctgcgtggtccaccacttttctcctaaccggggatttttttttttcttctgccactcttatctttccccacttcattccacccagtctccctcccccgtccctgcccaaacgcgcgcccctccgcccctcccttggccccagcgcccagccctgctctccgcgctcggccagagggagccagtccggagacggccgcacctggctggagaggctgggcgggcggaggggtggagacccgcggacgccgggaagccggacctggagccggagcagccgcgagcagaatggagtctcctaacagcctctcggtgctgatgtgaaatttgaccatctgattccagtttttttcttttccttttcttttttgcatttccttccctcgccatccgtcgtgtagtgaattgttcagtcttgctccgtttcaagagaggagatcatgattgagtgaagccaccccgtccgcagccaggaaaagcacaaagaagaaactgcaacaatggccaagctgacagaatccatgactaacgtcctggagggcgactccatggatcaggacgtcgaaagcccagtggccattcaccagccaaagttgcctaagcaggccagggatgacctgccaagacacatcagccgagatcggaccaaaaggaaaatccagaggtacgtgaggaaagacggaaagtgcaatgttcatcacggcaacgtgagggagacctatcgctacctgaccgatatcttcaccacattagtggacctgaagtggagattcaacctattgatttttgtcatggtttacacagtgacctggctcttttttggaatgatctggtggttgatcgcatacatacggggagacatggaccacatagaggacccctcctggactccttgtgttaccaacctcaacgggttcgtctctgcttttttattctcaatagagacagaaaccaccattggttatggctaccgggtcatcacagataaatgcccggagggaattattcttctcttaatccaatctgtgttggggtccattgtcaatgcattcatggtgggatgcatgtttgtaaaaatctctcaacccaagaagagggcagagaccctggtcttttccacccatgcagtgatctccatgcgggatgggaaactgtgcctgatgttccgggtaggggaccttaggaattcccacattgtggaggcttccatcagagccaagttgatcaaatccaaacagacctcggagggggagttcatcccgttgaaccagacggatatcaacgtagggtattacacgggggatgaccgtctgtttctggtgtcaccgctgatcattagccatgaaattaaccaacagagtcctttctgggagatctccaaagcccagctgcccaaagaggaactggaaattgtggtcatcctagaaggaatggtggaagccacagggatgacatgccaagctcgaagctcctacatcaccagtgagatcctgtggggttaccggttcacacctgtcctgaccctggaggatgggttctacgaagttgactacaacagcttccatgagacctatgagaccagcaccccatcccttagtgccaaagagctggccgagttagccagcagggcagagctgcccctgagttggtctgtatccagcaaactcaaccaacatgcagaactggagactgaagaggaagaaaagaacctcgaagagcaaacagaaagaaatggtgatgtggcaaacctggagaatgaatccaaagtttagtgccctagctgggcaaacccttctcttctccccccaacacaatctttccttgtctctcattctctttctttttctgtctctcttgctttgttctttatttgtttatatttaatttttacatgaccagaaaacaaatcttcaaggtgtaaaatatctacctgccctctctcagttattcagattgacaaggtagacatggatttgatgaaagtgcaaagtgccctcatttgtggcccaagcctggtctcctcccaaaatactacacatccaactcctggagatttcagttacttacctgcatgtgttgtacaataccagatcactcaaaaaggtgtgtcaaagattttacctgggatatgacaagcaaggtttctggtgcctatttattcattcagtgagacacagagtggagccctcagttttatggatcccaattcatttcatctactacagggtgaggtgcttgcccccatgtgggtgtggcagttacagggcccaggtgagctgaagacaaaccactgtacatatatatgccttatgtaattattttctttttgtaattagtaataaaacccagcatgtacaaaagtaccatagaacagaactgctaaatactgtacatagatgtatcattaatgtaggtttagatatataactttagaaataagaagcaaa

SEQ ID NO: 15 - DNA, 57 bp, Artificial Sequence
  Description of Artificial Sequencenucleotide sequence of the 57 bp KCNJ6 cDNA fragment
gcatttccttccctcgccatccgtcgtgtagtgaattgttcagtcttgctccgtatc

SEQ ID NO: 16 - DNA, 1272 bp, Homo sapiens
atggccaagctgacagaatccatgactaacgtcctggagggcgactccatggatcaggacgtcgaaagcccagtggccattcaccagccaaagttgcctaagcaggccagggatgacctgccaagacacatcagccgagatcggaccaaaaggaaaatccagaggtacgtgaggaaagacggaaagtgcaatgttcatcacggcaacgtgagggagacctatcgctacctgaccgatatcttcaccacattagtggacctgaagtggagattcaacctattgatttttgtcatggtttacacagtgacctggctcttttttggaatgatctggtggttgatcgcatacatacggggagacatggaccacatagaggacccctcctggactccttgtgttaccaacctcaacgggttcgtctctgcttttttattctcaatagagacagaaaccaccattggttatggctaccgggtcatcacagataaatgcccggagggaattattcttctcttaatccaatctgtgttggggtccattgtcaatgcattcatggtgggatgcatgtttgtaaaaatctctcaacccaagaagagggcagagaccctggtcttttccacccatgcagtgatctccatgcgggatgggaaactgtgcctgatgttccgggtaggggaccttaggaattcccacattgtggaggcttccatcagagccaagttgatcaaatccaaacagacctcggagggggagttcatcccgttgaaccagacggatatcaacgtagggtattacacgggggatgaccgtctgtttctggtgtcaccgctgatcattagccatgaaattaaccaacagagtcctttctgggagatctccaaagcccagctgcccaaagaggaactggaaattgtggtcatcctagaaggaatggtggaagccacagggatgacatgccaagctcgaagctcctacatcaccagtgagatcctgtggggttaccggttcacacctgtcctgaccctggaggatgggttctacgaagttgactacaacagcttccatgagacctatgagaccagcaccccatcccttagtgccaaagagctggccgagttagccagcagggcagagctgcccctgagttggtctgtatccagcaaactcaaccaacatgcagaactggagactgaagaggaagaaaagaacctcgaagagcaaacagaaagaaatggtgatgtggcaaacctggagaatgaatccaaagtttag